(19) United States
(12) Patent Application Publication
(10) Pub. No.: US 2012/0129189 A1
(43) Pub. Date: May 24, 2012

(54) Methods of Controlling Cell Proliferation

(76) Inventors: Chulan Kwon, Ellicott City, MD (US); Deepak Srivastava, San Francisco, CA (US)

(21) Appl. No.: 13/383,819

(22) PCT Filed: Jul. 16, 2010
(86) PCT No.: PCT/US10/42311

§ 371 Date: Feb. 9, 2012

(30) Foreign Application Priority Data
Jul. 17, 2009 (US) 61226588

(51) Int. Cl. G01N 33/567; C12Q 1/37; G01N 33/53; C12N 5/0789; C12N 15/85

(52) U.S. Cl. 435/7.21; 435/375; 435/455; 435/7.92; 435/24

(57) ABSTRACT

The present disclosure provides methods for increasing self-renewal/expansion of stem cells. The present disclosure provides methods of reducing uncontrolled cell proliferation. The present disclosure provides methods of identifying agents that modulate Notch1/-catenin binding, and methods of identifying agents that inhibit enzyme-mediated cleavage of Notch1 intracellular domain from Notch1 transmembrane domain.

### CROSS-REFERENCE

This application claims the benefit of U.S. Provisional Patent Application No. 61/226,588, filed Jul. 17, 2009, which application is incorporated herein by reference in its entirety.

### BACKGROUND

Stem cells hold tremendous therapeutic potential due to their unique ability to self-renew and to differentiate into specific cell types. Notch and Wnt/β-Catenin pathways are evolutionarily conserved signaling cascades pivotal for numerous cell-fate decision processes, including the binary decision of stem cell renewal or differentiation. Notch1 is a multi-functional transmembrane receptor that plays an important role in cellular differentiation. Binding of any one of the Notch ligands, such as Delta1 or Jagged1, to Notch1 results in activation of the Notch1 protein. The activated form of Notch1 then translocates to the nucleus and transactivates various target genes.

Canonical Wnt signals are mediated by the transcription factor, β-Catenin. In the absence of Wnt signaling, β-Catenin is phosphorylated by a destruction complex of glycogen synthase kinase-3β (GSK3β), adenomatous polyposis coli (APC), and axin. The phosphorylated β-Catenin is then specifically recognized and degraded by β-TrCP, a component of the ubiquitin ligase complex. Wnt signaling disrupts the destruction complex, allowing the unphosphorylated β-Catenin protein to accumulate and function as a co-activator for the transcription factor TCF/LEF. Human mutations in APC are associated with colon cancer due to excessive accumulation of β-Catenin activity in intestinal stem cells.

### LITERATURE

WO 2004/090110; WO 2006/052128; US Patent Publication No. 2008/0058316; van Es et al. (2005) Nature 435:959; Curry et al. (2005) Oncogene 24:6333; Shih et al. (2007) Cancer Res. 67:1879; Schroeter et al. (1998) Nature 393:382.

### SUMMARY OF THE INVENTION

The present disclosure provides methods for increasing self-renewal/expansion of stem cells. The present disclosure provides methods of reducing uncontrolled cell proliferation. The present disclosure provides methods of identifying agents that modulate Notch1/β-catenin binding, and methods of identifying agents that inhibit enzyme-mediated cleavage of Notch1 intracellular domain from Notch1 transmembrane domain.

### BRIEF DESCRIPTION OF THE DRAWINGS

FIGS. 1A-F depict Notch1 regulation of β-catenin in stem cells.

FIGS. 2A-J depict Notch1 regulation of β-catenin in embryonic stem cells through physical interaction with the Notch1 RAM domain.

FIGS. 3A-I depict the effect of membrane-bound Notch active β-catenin levels in stem cells.

FIGS. 4A and 4B depict the requirement for Numb and Numb-like for Notch-mediated regulation of β-catenin protein and activity.

FIGS. 5A-I depict the effect of γ-secretase inhibitors on expansion of human colon cancer cells and Notch cleavage.

FIG. 6 depicts a model for post-translational regulation of β-catenin protein.

FIGS. 7A and 7B depict an amino acid sequence of a Notch1 polypeptide.

FIG. 8 depicts an amino acid sequence of a RAM domain of a Notch1 polypeptide.

FIG. 9 depicts an amino acid sequence of a β-catenin polypeptide.

### DEFINITIONS

As used herein, the term “stem cell” refers to an undifferentiated cell that can be induced to proliferate. The stem cell is capable of self-maintenance, meaning that with each cell division, one daughter cell will also be a stem cell. Stem cells can be obtained from embryonic, fetal, post-natal, juvenile, or adult tissue. The term “progenitor cell”, as used herein, refers to an undifferentiated cell derived from a stem cell, and is not itself a stem cell. Some progenitor cells can produce progeny that are capable of differentiating into more than one cell type.

The term “induced pluripotent stem cell” (or “iPS cell”), as used herein, refers to a stem cell induced from a somatic cell, e.g., a differentiated somatic cell, and that has a higher potency than said somatic cell. iPS cells are capable of self-renewal and differentiation into mature cells.

The terms “polynucleotide” and “nucleic acid,” used interchangeably herein, refer to a polymeric form of nucleotides of any length, either ribonucleotides or deoxynucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The nucleic acid may be double stranded, single stranded, or contain portions of both double stranded or single stranded sequence. As will be appreciated by those in the art, the depiction of a single strand (“Watson”) also defines the sequence of the other strand (“Crick”). By the term “recombinant nucleic acid” herein is meant nucleic acid, originally formed in vitro, in general, by the manipulation of nucleic acid by endonucleases, in a form not normally found in nature. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention.

Nucleic acid sequence identity (as well as amino acid sequence identity) is calculated based on a reference sequence, which may be a subset of a larger sequence, such as a conserved motif, coding region, flanking region, etc. A reference sequence will usually be at least about 18 residues long, more usually at least about 30 residues long, and may extend to the complete sequence that is being compared. Algorithms for sequence analysis are known in the art, such as BLAST, described in Altschul et al. (1990), J. Mol. Biol. 215:403-10 (using default settings, i.e. parameters w=4 and T=17).

The terms “polypeptide,” “peptide,” and “protein,” used interchangeably herein, refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; and the like. NH₂ refers to the free amino group present at the amino terminus of a polypeptide. COOH refers to the free carboxyl group present at the carboxyl terminus of a polypeptide. In keeping with standard polypeptide nomenclature, J. Biol. Chem., 243 (1969), 3552-59 is used.

A “variant” of a polypeptide is defined as an amino acid sequence that is altered by one or more amino acids (e.g., by deletion, addition, insertion and/or substitution). Generally, “addition” refers to nucleotide or amino acid residues added to an end of the molecule, while “insertion” refers to nucleotide or amino acid residues between residues of a naturally-occurring molecule. The variant can have “conservative” changes, wherein a substituted amino acid has similar structural or chemical properties, e.g., replacement of leucine with isoleucine. More rarely, a variant can have “nonconservative” changes, e.g., replacement of a glycine with a tryptophan. Similar minor variations can also include amino acid deletions or insertions, or both. Guidance in determining which and how many amino acid residues may be substituted, added, inserted or deleted without abolishing biological or immunological activity can be found using computer programs well known in the art, for example, DNAStar software.

The term “genetic modification” and refers to a permanent or transient genetic change induced in a cell following introduction of new nucleic acid (i.e., nucleic acid exogenous to the cell). Genetic change (“modification”) can be accomplished by incorporation of the new nucleic acid into the genome of the host cell, or by transient or stable maintenance of the new nucleic acid as an extrachromosomal element. Where the cell is a eukaryotic cell, a permanent genetic change can be achieved by introduction of the nucleic acid into the genome of the cell. Suitable methods of genetic modification include viral infection, transfection, conjugation, protoplast fusion, electroporation, particle gun technology, calcium phosphate precipitation, direct microinjection, and the like.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells. An isolated polypeptide will in some embodiments be synthetic. “Synthetic polypeptides” are assembled from amino acids, and are chemically synthesized in vitro, e.g., cell-free chemical synthesis, using procedures known to those skilled in the art.

By “purified” is meant a compound of interest (e.g., a polypeptide) has been separated from components that accompany it in nature. “Purified” can also be used to refer to a compound of interest separated from components that can accompany it during manufacture (e.g., in chemical synthesis). In some embodiments, a compound is substantially pure when it is at least 50% to 60%, by weight, free from organic molecules with which it is naturally associated or with which it is associated during manufacture. In some embodiments, the preparation is at least 75%, at least 90%, at least 95%, or at least 99%, by weight, of the compound of interest. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample having a compound that binds an antibody of interest. Purity can be measured by any appropriate method, e.g., chromatography, mass spectroscopy, high performance liquid chromatography analysis, etc.

The terms “cancer, “neoplasm,” and “tumor” are used interchangeably herein to refer to cells which exhibit relatively autonomous growth, so that they exhibit an aberrant growth phenotype characterized by a significant loss of control of cell proliferation. Cells of interest that may exhibit uncontrolled proliferation include precancerous, malignant, pre-metastatic, metastatic, and non-metastatic cells, as well as carcinoma in situ.

The terms “individual,” “subject,” “host,” and “patient,” used interchangeably herein, refer to a mammal, including, but not limited to, murines (rats, mice), non-human primates, humans, canines, felines, ungulates (e.g., equines, bovines, ovines, porcines, caprines), etc. In some embodiments, the individual is a human. In some embodiments, the individual is a murine.

The terms “treatment,” “treating,” “treat,” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

A “therapeutically effective amount” or “efficacious amount” means the amount of an agent that, when administered to a mammal or other subject for treating a disease, is sufficient to effect such treatment for the disease. The “therapeutically effective amount” will vary depending on agent, the disease or condition and its severity and the age, weight, etc., of the subject to be treated.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a stem cell” includes a plurality of such stem cells and reference to “the Notch1 polypeptide” includes reference to one or more Notch1 polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods for increasing self-renewal/expansion of stem cells. The present disclosure provides methods of reducing uncontrolled cell proliferation. The present disclosure provides methods of identifying agents that modulate Notch1/β-catenin binding, and methods of identifying agents that inhibit enzyme-mediated cleavage of Notch1 intracellular domain from Notch1 transmembrane domain.

The present disclosure is based in part on the observation that the intracellular domain of membrane-bound Notch1 physically interacts with β-catenin; binding of Notch1 to β-catenin via the Notch1 intracellular domain (NICD) results in degradation of β-catenin.

### Methods for In Vitro Expansion of a Stem Cell

The present disclosure provides an in vitro method for increasing self-renewal or expansion of a stem cell. The method generally involves contacting a stem cell in vitro with an effective amount of an agent that inhibits binding between the intracellular domain of a membrane-bound Notch1 polypeptide and a β-catenin polypeptide. Stem cells are useful in a variety of treatment and research applications.

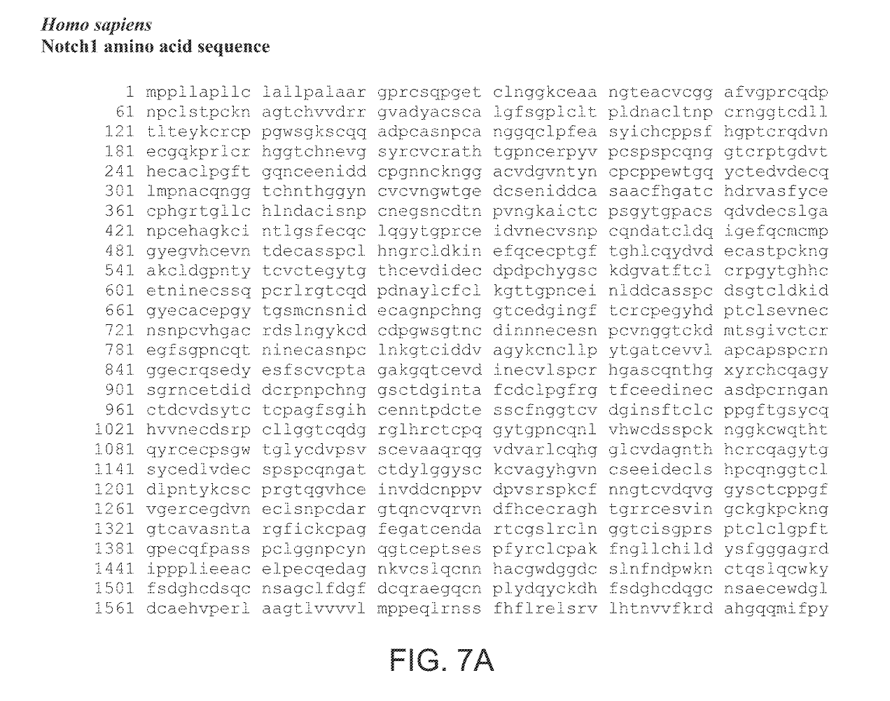

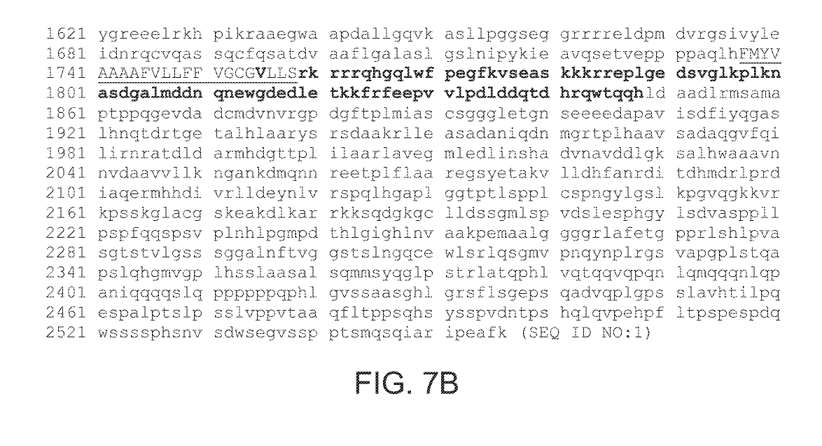

A membrane-bound Notch1 polypeptide can comprise an amino acid sequence having at least about 75%, at least about 80%, at least about 85%, at least about 90%, at least about 95%, at least about 98%, at least about 99%, or 100%, amino acid sequence identity with a contiguous stretch of from about 500 amino acids (aa) to about 750 aa, from about 750 aa to about 1000 aa, from about 1000 aa to about 1500 aa, from about 1500 aa to about 1750 aa, from about 1750 aa to about 2000 aa, from about 2000 aa to about 2250 aa, or from about 2250 aa to 2556 aa of the amino acid sequence set forth in SEQ ID NO:1 and depicted in FIGS. 7A and 7B.

A β-catenin polypeptide can comprise an amino acid sequence having at least about 75%, at least about 80%, at least about 85%, at least about 90%, at least about 95%, at least about 98%, at least about 99%, or 100%, amino acid sequence identity with a contiguous stretch of from about 400 aa to about 500 aa, from about 500 aa to about 600 aa, from about 600 aa to about 700 aa, or from about 700 aa to 781 aa of the amino acid sequence set forth in SEQ ID NO:3 and depicted in FIG. 9.

Suitable stem cells include embryonic stem cells, post-natal stem cells, adult stem cells, and induced pluripotent stem (iPS) cells. Suitable stem cells include, e.g., cardiac stem cells, mesenchymal stem cells, hematopoietic stem cells, neural stem cells, and the like.

iPS cells are generated from mammalian cells (including mammalian somatic cells) using, e.g., known methods. Examples of suitable mammalian cells include, but are not limited to: fibroblasts, skin fibroblasts, dermal fibroblasts, bone marrow-derived mononuclear cells, skeletal muscle cells, adipose cells, peripheral blood mononuclear cells, macrophages, hepatocytes, keratinocytes, oral keratinocytes, hair follicle dermal cells, epithelial cells, gastric epithelial cells, lung epithelial cells, synovial cells, kidney cells, skin epithelial cells, pancreatic beta cells, and osteoblasts.

Mammalian cells used to generate iPS cells can originate from a variety of types of tissue including but not limited to: bone marrow, skin (e.g., dermis, epidermis), muscle, adipose tissue, peripheral blood, foreskin, skeletal muscle, and smooth muscle. The cells used to generate iPS cells can also be derived from neonatal tissue, including, but not limited to: umbilical cord tissues (e.g., the umbilical cord, cord blood, cord blood vessels), the amnion, the placenta, and various other neonatal tissues (e.g., bone marrow fluid, muscle, adipose tissue, peripheral blood, skin, skeletal muscle etc.).

Cells used to generate iPS cells can be derived from tissue of a non-embryonic subject, a neonatal infant, a child, or an adult. Cells used to generate iPS cells can be derived from neonatal or post-natal tissue collected from a subject within the period from birth, including cesarean birth, to death. For example, the tissue source of cells used to generate iPS cells can be from a subject who is greater than about 10 minutes old, greater than about 1 hour old, greater than about 1 day old, greater than about 1 month old, greater than about 2 months old, greater than about 6 months old, greater than about 1 year old, greater than about 2 years old, greater than about 5 years old, greater than about 10 years old, greater than about 15 years old, greater than about 18 years old, greater than about 25 years old, greater than about 35 years old, >45 years old, >55 years old, >65 years old, >80 years old, <80 years old, <70 years old, <60 years old, <50 years old, <40 years old, <30 years old, <20 years old or <10 years old.

iPS cells produce and express on their cell surface one or more of the following cell surface antigens: SSEA-3, SSEA-4, TRA-1-60, TRA-1-81, TRA-2-49/6E (alkaline phophatase), and Nanog. In some embodiments, iPS cells produce and express on their cell surface SSEA-3, SSEA-4, TRA-1-60, TRA-1-81, TRA-2-49/6E, and Nanog. iPS cells express one or more of the following genes: Oct-3/4, Sox2, Nanog, GDF3, REX1, FGF4, ESG1, DPPA2, DPPA4, and hTERT. In some embodiments, an iPS cell expresses Oct-3/4, Sox2, Nanog, GDF3, REX1, FGF4, ESG1, DPPA2, DPPA4, and hTERT.

Methods of generating iPS cells are known in the art, and a wide range of methods can be used to generate iPS cells. See, e.g., Takahashi and Yamanaka (2006) Cell 126:663-676; Yamanaka et al. (2007) Nature 448:313-7; Wernig et al. (2007) Nature 448:318-24; Maherali (2007) Cell Stem Cell 1:55-70; Maherali and Hochedlinger (2008) Cell Stem Cell 3:595-605; Park et al. (2008) Cell 134:1-10; Dimos et. al. (2008) Science 321:1218-1221; Blelloch et al. (2007) Cell Stem Cell 1:245-247; Stadtfeld et al. (2008) Science 322:945-949; Stadtfeld et al. (2008) 2:230-240; Okita et al. (2008) Science 322:949-953.

In some embodiments, iPS cells are generated from somatic cells by forcing expression of a set of factors in order to promote increased potency of a cell or de-differentiation. Forcing expression can include introducing expression vectors encoding polypeptides of interest into cells, introducing exogenous purified polypeptides of interest into cells, or contacting cells with a reagent that induces expression of an endogenous gene encoding a polypeptide of interest.

Forcing expression may include introducing expression vectors into somatic cells via use of moloney-based retroviruses (e.g., moloney leukemia virus; MLV), lentiviruses (e.g., human immunodeficiency virus; HIV), adenoviruses, protein transduction, transient transfection, or protein transduction. In some embodiments, the moloney-based retroviruses or HIV-based lentiviruses are pseudotyped with envelope from another virus, e.g. vesicular stomatitis virus-g (VSV-g) using known methods in the art. See, e.g. Dimos et al. (2008) Science 321:1218-1221.

In some embodiments, iPS cells are generated from somatic cells by forcing expression of Oct-3/4 and Sox2 polypeptides. In some embodiments, iPS cells are generated from somatic cells by forcing expression of Oct-3/4, Sox2 and Klf4 polypeptides. In some embodiments, iPS cells are generated from somatic cells by forcing expression of Oct-3/4, Sox2, Klf4 and c-Myc polypeptides. In some embodiments, iPS cells are generated from somatic cells by forcing expression of Oct-4, Sox2, Nanog, and LIN28 polypeptides.

For example, iPS cells can be generated from somatic cells by genetically modifying the somatic cells with one or more expression constructs encoding Oct-3/4 and Sox2. As another example, iPS cells can be generated from somatic cells by genetically modifying the somatic cells with one or more expression constructs comprising nucleotide sequences encoding Oct-3/4, Sox2, c-myc, and Klf4. As another example, iPS cells can be generated from somatic cells by genetically modifying the somatic cells with one or more expression constructs comprising nucleotide sequences encoding Oct-4, Sox2, Nanog, and LIN28.

In some embodiments, cells undergoing induction of pluripotency as described above, to generate iPS cells, are contacted with additional factors which can be added to the culture system, e.g., included as additives in the culture medium. Examples of such additional factors include, but are not limited to: histone deacetylase (HDAC) inhibitors, see, e.g. Huangfu et al. (2008) Nature Biotechnol. 26:795-797; Huangfu et al. (2008) Nature Biotechnol. 26: 1269-1275; DNA demethylating agents, see, e.g., Mikkelson et al (2008) Nature 454, 49-55; histone methyltransferase inhibitors, see, e.g., Shi et al. (2008) Cell Stem Cell 2:525-528; L-type calcium channel agonists, see, e.g., Shi et al. (2008) 3:568-574; Wnt3a, see, e.g., Marson et al. (2008) Cell 134:521-533; and short interfering RNA (siRNA), see, e.g., Zhao et al. (2008) Cell Stem Cell 3: 475-479.

In some embodiments, iPS cells are generated from somatic cells by forcing expression of Oct3/4, Sox2 and contacting the cells with an HDAC inhibitor, e.g., valproic acid. See, e.g., Huangfu et al. (2008) Nature Biotechnol. 26: 1269-1275. In some embodiments, iPS cells are generated from somatic cells by forcing expression of Oct3/4, Sox2, and Klf4 and contacting the cells with an HDAC inhibitor, e.g., valproic acid. See, e.g., Huangfu et al. (2008) Nature Biotechnol. 26:795-797.

In some embodiments, an effective amount of an agent that inhibits binding between the intracellular domain of a membrane-bound Notch1 polypeptide and a β-catenin polypeptide is an amount of an agent that inhibits binding of the intracellular domain of a membrane-bound Notch1 polypeptide to a β-catenin polypeptide by at least about 10%, at least about 15%, at least about 20%, at least about 25%, at least about 30%, at least about 40%, at least about 50%, at least about 60%, at least about 70%, at least about 80%, at least about 90%, or more than 90%, compared to the binding between the intracellular domain of the membrane-bound Notch1 polypeptide and the β-catenin polypeptide in the absence of the agent.

An agent that inhibits binding between the intracellular domain of a membrane-bound Notch1 polypeptide and a β-catenin polypeptide can: 1) reduce degradation of β-catenin; and 2) increase self-renewal/expansion of a stem cell.

In some embodiments, an agent that inhibits binding between the intracellular domain of a membrane-bound Notch1 polypeptide and a β-catenin polypeptide reduces degradation of β-catenin polypeptides in a stem cell by at least about 10%, at least about 15%, at least about 20%, at least about 25%, at least about 30%, at least about 40%, at least about 50%, at least about 60%, at least about 70%, at least about 80%, at least about 90%, or more than 90%, compared to the level of degradation of the β-catenin polypeptide population in the stem cell in the absence of the agent.

In some embodiments, an agent that inhibits binding between the intracellular domain of a membrane-bound Notch1 polypeptide and a β-catenin polypeptide increases self-renewal or expansion of a stem cell, such that the number of stem cells in a population of stem cells contacted with the agent increases over a given period of time by at least about 25%, at least about 50%, at least about 75%, at least about 2-fold, at least about 5-fold, at least about 10-fold, at least about 50-fold, or at least about 100-fold, or more than 100-fold, than the increase in the number of stem cells in the absence of the agent over the same time period. In other words, the rate of increase in the number of stem cells in a stem cell population is by at least about 25%, at least about 50%, at least about 75%, at least about 2-fold, at least about 5-fold, at least about 10-fold, at least about 50-fold, or at least about 100-fold, or more than 100-fold, greater when the stem cells are contacted with an agent that inhibits binding between the intracellular domain of a membrane-bound Notch1 polypeptide and a β-catenin polypeptide, compared to the rate of increase in the number of stem cells in a control stem cell population not contacted with the agent.

Suitable agents include, e.g., a polypeptide fragment of Notch1 intracellular domain (NICD) that competes with full-length Notch1 for binding to β-catenin, where the polypeptide fragment does not induce degradation of β-catenin. Such Notch1 fragments are referred to herein as “competitive inhibitor Notch1 fragments.” Exemplary suitable competitive inhibitor Notch1 fragments include, but are not limited to, a RAM domain fragment; an ankyrin-like repeat fragment; a transactivation domain fragment; a PEST domain fragment; and a fragment that overlaps or otherwise includes all or part of one or more of a RAM domain, an ankyrin-like repeat, a transactivation domain, and a PEST domain of a NICD.

A suitable competitive inhibitor Notch1 fragment includes, e.g., a polypeptide having a length of from about 20 amino acids to about 25 aa, from about 25 aa to about 30 aa, from about 30 aa to about 40 aa, from about 40 aa to about 50 aa, from about 50 aa to about 75 aa, from about 75 aa to about 100 aa, from about 100 aa to about 200 aa, from about 200 aa to about 300 aa, from about 300 aa to about 400 aa, from about 400 aa to about 500 aa, from about 500 aa to about 600 aa, from about 600 aa to about 700 aa, or from about 700 aa to about 800 aa, of a NICD. For example, a suitable competitive inhibitor Notch1 fragment can have an amino acid sequence that has at least about 75%, at least about 80%, at least about 85%, at least about 90%, at least about 95%, at least about 98%, at least about 99%, or 100%, amino acid sequence identity with a contiguous stretch of from about 20 amino acids to about 25 aa, from about 25 aa to about 30 aa, from about 30 aa to about 40 aa, from about 40 aa to about 50 aa, from about 50 aa to about 75 aa, from about 75 aa to about 100 aa, from about 100 aa to about 200 aa, from about 200 aa to about 300 aa, from about 300 aa to about 400 aa, from about 400 aa to about 500 aa, from about 500 aa to about 600 aa, from about 600 aa to about 700 aa, or from about 700 aa to 798 aa, of amino acids 1759-2556 of the amino acid sequence set forth in SEQ ID NO:1 and depicted in FIGS. 7A and 7B.

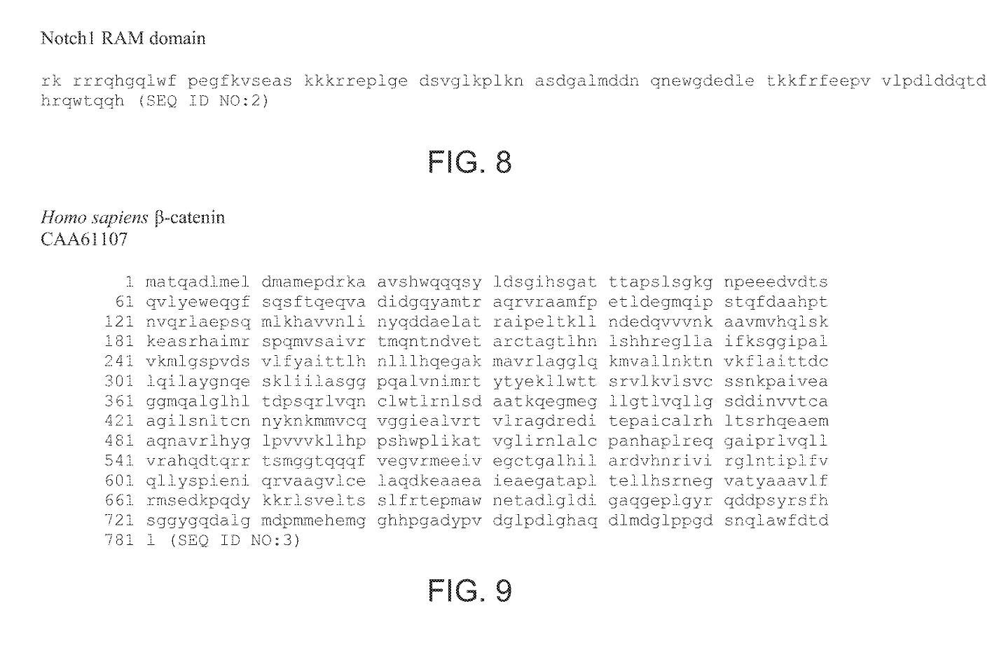

In some embodiments, a suitable competitive inhibitor Notch1 fragment comprises a RAM domain of a Notch1 polypeptide. For example, in some embodiments, a suitable competitive inhibitor Notch1 fragment comprises an amino acid sequence having at least about 75%, at least about 80%, at least about 85%, at least about 90%, at least about 95%, at least about 98%, at least about 99%, or 100%, amino acid sequence identity with a contiguous stretch of from about 20 amino acids to about 25 aa, from about 25 aa to about 30 aa, from about 30 aa to about 40 aa, from about 40 aa to about 50 aa, from about 50 aa to about 75 aa, or from about 75 aa to 90 aa, of the amino acid sequence set forth in SEQ ID NO:2 and depicted in FIG. 8.

As another example, in some embodiments, a suitable competitive inhibitor Notch1 fragment has a length of from about 15 aa to about 20 aa, from about 20 aa to about 25 aa, from about 25 aa to about 30 aa, from about 30 aa to about 35 aa, from about 35 aa to about 40 aa, from about 40 aa to about 50 aa, from about 50 aa to about 60 aa, from about 60 aa to about 70 aa, from about 70 aa to about 80 aa, from about 80 aa to about 90 aa, or from about 90 aa to about 100 aa, and comprises an amino acid sequence having at least about 75%, at least about 80%, at least about 85%, at least about 90%, at least about 95%, at least about 98%, at least about 99%, or 100%, amino acid sequence identity with a contiguous stretch of from about 20 amino acids to about 25 aa, from about 25 aa to about 30 aa, from about 30 aa to about 40 aa, from about 40 aa to about 50 aa, from about 50 aa to about 75 aa, or from about 75 aa to 90 aa, of the amino acid sequence set forth in SEQ ID NO:2 and depicted in FIG. 8.

In some embodiments, a suitable competitive inhibitor Notch1 fragment comprises all or a portion of an ankyrin-repeat domain of a Notch1 polypeptide. For example, comprises an amino acid sequence having at least about 75%, at least about 80%, at least about 85%, at least about 90%, at least about 95%, at least about 98%, at least about 99%, or 100%, amino acid sequence identity with a contiguous stretch of from about 25 aa to about 50 aa, from about 50 aa to about 100 aa, from about 100 aa to about 200 aa, from about 200 aa to about 300 aa, from about 300 aa to about 400 aa, from about 400 aa to about 500 aa, from about 500 aa to about 600 aa, from about 600 aa to about 700 aa, from about 700 aa to about 800 aa, from about 800 aa to about 900 aa, or from about 900 aa to about 1000 aa, of amino acids 1922 to 2034, or amino acids 1989 to 2113, of the amino acid sequence set forth in SEQ ID NO:1 and depicted in FIGS. 7A and 7B.

In some embodiments, a suitable competitive inhibitor Notch1 fragment has 1, 2, 3, 4, 5, 6, 7, 8, 9, 10, 10-15, or 10-15 amino acid substitutions compared to a wild-type NICD, such that the fragment competitively inhibits binding between an NICD and a β-catenin polypeptide, but does not induce degradation of the β-catenin polypeptide.

In some embodiments, a competitive inhibitor Notch1 fragment is synthetic. In some embodiments, a competitive inhibitor Notch1 fragment is cyclic. In some embodiments, a competitive inhibitor Notch1 fragment comprises one or more modifications such as: 1) a poly(ethylene glycol) (PEG) moiety; 2) a saccharide moiety; 3) a carbohydrate moiety; 4) a myristyl group; 5) a lipid moiety; and the like.

In some embodiments, a suitable competitive inhibitor Notch1 fragment is cyclized. Methods of cyclizing a peptide are known in the art, and any of a variety of established methods can be used to cyclize a peptide. For example, a peptide can be synthesized to include a Cys at or near the amino terminus and a Cys at or near the carboxyl terminus, and a disulfide bond can be formed between the two Cys residues.

In some embodiments, a suitable competitive inhibitor Notch1 fragment comprises a protein transduction domain. “Protein Transduction Domain” or PTD refers to a polypeptide, polynucleotide, carbohydrate, or organic or inorganic compound that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of a competitive inhibitor Notch1 fragment. In some embodiments, a PTD is covalently linked to the carboxyl terminus of a competitive inhibitor Notch1 fragment.

Exemplary protein transduction domains include but are not limited to a minimal undecapeptide protein transduction domain (corresponding to residues 47-57 of HIV-1 TAT comprising YGRKKRRQRRR; SEQ ID NO:4); a polyarginine sequence comprising a number of arginines sufficient to direct entry into a cell (e.g., 3, 4, 5, 6, 7, 8, 9, 10, or 10-50 arginines); a VP22 domain (Zender et al., Cancer Gene Ther. 2002 June; 9(6):489-96); an Drosophila Antennapedia protein transduction domain (Noguchi et al., Diabetes 2003; 52(7):1732-1737); a truncated human calcitonin peptide (Trehin et al. Pharm. Research, 21:1248-1256, 2004); polylysine (Wender et al., PNAS, Vol. 97:13003-13008); RRQRRTSKLMKR (SEQ ID NO:5); Transportan GWTLNSAGYLLGKINLKALAALAKKIL (SEQ ID NO:6); KALAWEAKLAKALAKALAKHLAKALAKALKCEA (SEQ ID NO:7); and RQIKIWFQNRRMKWKK (SEQ ID NO:8). Exemplary PTDs include but are not limited to, YGRKKRRQRRR (SEQ ID NO:9), RKKRRQRRR (SEQ ID NO:10); an arginine homopolymer of from 3 arginine residues to 50 arginine residues; Exemplary PTD domain amino acid sequences include, but are not limited to, any of the following: YARAAARQARA (SEQ ID NO:11); THRLPRRRRRR (SEQ ID NO:12); and GGRRARRRRRR (SEQ ID NO:13).

In some embodiments, a competitive inhibitor Notch1 fragment polypeptide is introduced into a stem cell. In other embodiments, a nucleic acid (e.g., an expression vector) comprising a nucleotide sequence encoding a competitive inhibitor Notch1 fragment is introduced into a stem cell, where the encoded competitive inhibitor Notch1 fragment is synthesized in the stem cell.

Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:8186, 1998, Flannery et al., PNAS 94:6916 6921, 1997; Bennett et al., Invest Opthalmol V is Sci 38:2857 2863, 1997; Jomary et al., Gene Ther 4:683 690, 1997, Rolling et al., Hum Gene Ther 10:641648, 1999; Ali et al., Hum Mol Genet. 5:591594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

Numerous suitable expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other vector may be used so long as it is compatible with the host cell.

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544). For example, in some embodiments, a nucleotide sequence encoding a competitive inhibitor Notch1 fragment can be operably linked to a promoter, which may be constitutive or inducible. Non-limiting examples of suitable eukaryotic promoters (promoters functional in a eukaryotic cell) include cytomegalovirus immediate early promoter, herpes simplex virus thymidine kinase promoter, early and late SV40 promoter, long terminal repeats (LTRs) from retrovirus, and mouse metallothionein-I promoter; and inducible promoters, such as those containing Tet-operator elements.

In some cases, the expression vector(s) encodes, in addition to a competitive inhibitor Notch1 fragment, a marker gene that facilitates identification or selection of cells that have been transfected or infected. Examples of marker genes include, but are not limited to, genes encoding fluorescent proteins, e.g., enhanced green fluorescent protein, Ds-Red (DsRed: Discosoma sp. red fluorescent protein (RFP); Bevis and Glick (2002) Nat. Biotechnol. 20:83), yellow fluorescent protein, and cyanofluorescent protein; and genes encoding proteins conferring resistance to a selection agent, e.g., a neomycin resistance gene, a puromycin resistance gene, a blasticidin resistance gene, and the like.

The present disclosure further provides an isolated (including synthetic) competitive inhibitor Notch1 fragment; compositions comprising an isolated competitive inhibitor Notch1 fragment; nucleic acids comprising nucleotide sequences encoding a competitive inhibitor Notch1 fragment; and compositions comprising nucleic acids comprising nucleotide sequences encoding a competitive inhibitor Notch1 fragment.

A subject composition can comprise: i) an isolated competitive inhibitor Notch1 fragment; and ii) one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES),2-(N-Morpholino)ethanesulfonic acid (MES),2-(N-Morpholino)ethanesulfonic acid sodium salt (MES),3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

### Methods of Reducing Uncontrolled Cell Proliferation

The present disclosure provides methods of reducing uncontrolled cell proliferation. The methods generally involve contacting a cell that exhibits uncontrolled cell proliferation (e.g., a cancer cell) with an agent that inhibits cleavage of a Notch1 intracellular domain (NICD) polypeptide from the transmembrane domain of the Notch1 polypeptide.

Uncontrolled cell proliferation occurs in various contexts, including cancer. In some embodiments, in the context of cancer treatment, an “effective amount” of an agent that inhibits cleavage of a NICD polypeptide from the transmembrane domain of Notch1 is an amount that, when administered to an individual in one or more doses, reduces one or more of tumor size, cancer cell number, and cancer cell metastasis by at least about 10%, at least about 20%, at least about 30%, at least about 40%, at least about 50%, at least about 60%, at least about 70%, at least about 80%, or at least about 90%, up to total eradication of the tumor.

A subject method is useful for treating a wide variety of cancers, including carcinomas, sarcomas, leukemias, and lymphomas.

Carcinomas that can be treated using a subject method include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

Sarcomas that can be treated using a subject method include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Other solid tumors that can be treated using a subject method include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

Leukemias that can be treated using a subject method include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a subject method include, but are not limited to, B-cell lymphomas (e.g., Burkitt's lymphoma); Hodgkin's lymphoma; and the like.

In some embodiments, an agent that inhibits cleavage of a NICD polypeptide from the transmembrane domain of Notch1 is administered as an adjuvant therapy to a standard cancer therapy. Standard cancer therapies include surgery (e.g., surgical removal of cancerous tissue), radiation therapy, bone marrow transplantation, chemotherapeutic treatment, biological response modifier treatment, and certain combinations of the foregoing.

Radiation therapy includes, but is not limited to, x-rays or gamma rays that are delivered from either an externally applied source such as a beam, or by implantation of small radioactive sources.

Chemotherapeutic agents are non-peptidic (i.e., non-proteinaceous) compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (vinca) alkaloids, and steroid hormones.

Agents that act to reduce cellular proliferation are known in the art and widely used. Such agents include alkylating agents, such as nitrogen mustards, nitrosoureas, ethylenimine derivatives, alkyl sulfonates, and triazenes, including, but not limited to, mechlorethamine, cyclophosphamide (Cytoxan™), melphalan (L-sarcolysin), carmustine (BCNU), lomustine (CCNU), semustine (methyl-CCNU), streptozocin, chlorozotocin, uracil mustard, chlormethine, ifosfamide, chlorambucil, pipobroman, triethylenemelamine, triethylenethiophosphoramine, busulfan, dacarbazine, and temozolomide.

Antimetabolite agents include folic acid analogs, pyrimidine analogs, purine analogs, and adenosine deaminase inhibitors, including, but not limited to, cytarabine (CYTOSAR-U), cytosine arabinoside, fluorouracil (5-FU), floxuridine (FudR),6-thioguanine, 6-mercaptopurine (6-MP), pentostatin, 5-fluorouracil (5-FU), methotrexate, 10-propargyl-5,8-dideazafolate (PDDF, CB3717), 5,8-dideazatetrahydrofolic acid (DDATHF), leucovorin, fludarabine phosphate, pentostatine, and gemcitabine.

Suitable natural products and their derivatives, (e.g., vinca alkaloids, antitumor antibiotics, enzymes, lymphokines, and epipodophyllotoxins), include, but are not limited to, Ara-C, paclitaxel (Taxol®), docetaxel (Taxotere®), deoxycoformycin, mitomycin-C, L-asparaginase, azathioprine; brequinar; alkaloids, e.g. vincristine, vinblastine, vinorelbine, vindesine, etc.; podophyllotoxins, e.g. etoposide, teniposide, etc.; antibiotics, e.g. anthracycline, daunorubicin hydrochloride (daunomycin, rubidomycin, cerubidine), idarubicin, doxorubicin, epirubicin and morpholino derivatives, etc.; phenoxizone biscyclopeptides, e.g. dactinomycin; basic glycopeptides, e.g. bleomycin; anthraquinone glycosides, e.g. plicamycin (mithramycin); anthracenediones, e.g. mitoxantrone; azirinopyrrolo indolediones, e.g. mitomycin; macrocyclic immunosuppressants, e.g. cyclosporine, FK-506 (tacrolimus, prograf), rapamycin, etc.; and the like.

Other anti-proliferative cytotoxic agents are navelbene, CPT-11, anastrazole, letrazole, capecitabine, reloxafine, cyclophosphamide, ifosamide, and droloxafine.

Microtubule affecting agents that have antiproliferative activity are also suitable for use and include, but are not limited to, allocolchicine (NSC 406042), Halichondrin B (NSC 609395), colchicine (NSC 757), colchicine derivatives (e.g., NSC 33410), dolstatin 10 (NSC 376128), maytansine (NSC 153858), rhizoxin (NSC 332598), paclitaxel (Taxol®), Taxol® derivatives, docetaxel (Taxotere®), thiocolchicine (NSC 361792), trityl cysterin, vinblastine sulfate, vincristine sulfate, natural and synthetic epothilones including but not limited to, eopthilone A, epothilone B, discodermolide; estramustine, nocodazole, and the like.

Hormone modulators and steroids (including synthetic analogs) that are suitable for use include, but are not limited to, adrenocorticosteroids, e.g. prednisone, dexamethasone, etc.; estrogens and pregestins, e.g. hydroxyprogesterone caproate, medroxyprogesterone acetate, megestrol acetate, estradiol, clomiphene, tamoxifen; etc.; and adrenocortical suppressants, e.g. aminoglutethimide; 17α-ethinylestradiol; diethylstilbestrol, testosterone, fluoxymesterone, dromostanolone propionate, testolactone, methylprednisolone, methyl-testosterone, prednisolone, triamcinolone, chlorotrianisene, hydroxyprogesterone, aminoglutethimide, estramustine, medroxyprogesterone acetate, leuprolide, Flutamide (Drogenil), Toremifene (Fareston), and Zoladex®. Estrogens stimulate proliferation and differentiation; therefore, compounds that bind to the estrogen receptor are used to block this activity. Corticosteroids may inhibit T cell proliferation.

Other chemotherapeutic agents include metal complexes, e.g. cisplatin (cis-DDP), carboplatin, etc.; ureas, e.g. hydroxyurea; and hydrazines, e.g. N-methylhydrazine; epidophyllotoxin; a topoisomerase inhibitor; procarbazine; mitoxantrone; leucovorin; tegafur; etc. Other anti-proliferative agents of interest include immunosuppressants, e.g. mycophenolic acid, thalidomide, desoxyspergualin, azasporine, leflunomide, mizoribine, azaspirane (SKF 105685); Iressa® (ZD 1839, 4-(3-chloro-4-fluorophenylamino)-7-methoxy-6-(3-(4-morpholinyl)propoxy)quinazoline); etc.

“Taxanes” include paclitaxel, as well as any active taxane derivative or pro-drug. “Paclitaxel” (which should be understood herein to include analogues, formulations, and derivatives such as, for example, docetaxel, TAXOL™, TAXOTERE™ (a formulation of docetaxel), 10-desacetyl analogs of paclitaxel and 3′N-desbenzoyl-3′N-t-butoxycarbonyl analogs of paclitaxel) may be readily prepared utilizing techniques known to those skilled in the art (see also WO 94/07882, WO 94/07881, WO 94/07880, WO 94/07876, WO 93/23555, WO 93/10076; U.S. Pat. Nos. 5,294,637; 5,283,253; 5,279,949; 5,274,137; 5,202,448; 5,200,534; 5,229,529; and EP 590,267), or obtained from a variety of commercial sources, including for example, Sigma Chemical Co., St. Louis, Mo. (T7402 from Taxus brevifolia; or T-1912 from Taxus yannanensis).

Paclitaxel should be understood to refer to not only the common chemically available form of paclitaxel, but analogs and derivatives (e.g., Taxotere™ docetaxel, as noted above) and paclitaxel conjugates (e.g., paclitaxel-PEG, paclitaxel-dextran, or paclitaxel-xylose).

Also included within the term “taxane” are a variety of known derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in WO 99/09021, WO 98/22451, and U.S. Pat. No. 5,869,680; 6-thio derivatives described in WO 98/28288; sulfenamide derivatives described in U.S. Pat. No. 5,821,263; and taxol derivative described in U.S. Pat. No. 5,415,869. It further includes prodrugs of paclitaxel including, but not limited to, those described in WO 98/58927; WO 98/13059; and U.S. Pat. No. 5,824,701.

### Agents

Agents that inhibit cleavage of a NICD polypeptide from the transmembrane domain of the Notch1 polypeptide include γ-secretase inhibitors.

Gamma-secretase inhibitors (GSI) that are suitable for use include those described in, e.g., U.S. Pat. No. 5,703,129; U.S. Pat. No. 6,448,229; U.S. Pat. No. 6,683,091; U.S. Pat. No. 6,756,511; U.S. Pat. No. 6,890,956; U.S. Pat. No. 6,984,626; U.S. Pat. No. 6,995,155; WO 01/70677; WO 02/081435; WO 03/018543; WO 00/50391; WO 03/0422646; WO 03/041735; and U.S. published applications 2005/0227973, 2006/0030694, 2006/0004004, 2006/0009467, 2005/0261276, 2005/0143369, and 2005/0075320. Also suitable for use are γ-secretase inhibitors described in, e.g., U.S. Patent Publication Nos. 2009/0118289, 2009/0105345, 2009/0105275, and 2008/0058316. Suitable GSIs include, e.g., a compound of any of Formulas I-XV, as described below.

For example, in some embodiments, a suitable GSI is a benzodiazepine derivative as described in U.S. Pat. No. 6,995,155, e.g., a compound of Formula I:

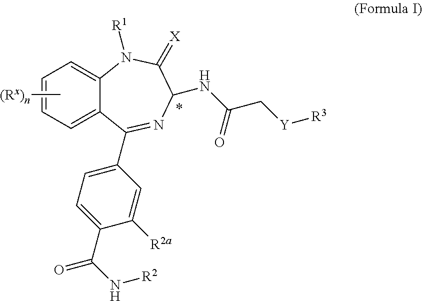

where n is 0, 1, 2 or 3; each R^{X} independently selected from a halogen, —CN, —NO₂, a C₁₋₆alkyl, a polyfluoroC₁₋₆alkyl, —OH and a C₁₋₄alkoxy;
X is O, S or N—R^{a} where optionally R^{a} together with R¹ completes a fused imidazole or 4,5-dihydroimidazole ring;

### Y is —CH₂—, —CH(OH)—, —CH(CH₃)—, —CH₂O—, —O— or —S;

R¹ is H, a C₁₋₆alkyl, a C₃₋₈cycloalkyl, a C₂₋₆alkenyl, a C₂₋₆alkynyl or a polyfluoroC₁₋₆alkyl, where the alkyl, cycloalkyl, alkenyl and alkynyl groups are optionally substituted with a halogen, —CN, —NO₂, an aryl, a heteroaryl, —COR⁶, —CO₂R⁶, —CON(R⁶)₂, —OCOR⁷, —NR⁶COR⁷, —NR⁶SO₂R⁷, —SO₃R⁶, —SO₂N(R⁶)₂, —OR⁶, —SR⁶ or —N(R⁶)₂; or when X is N—R^{a}, optionally R¹ together with R^{a} completes a fused imidazole or 4,5-dihydroimidazole ring;
R² and R^{2a} each represents hydrogen, or R² and R^{2a} together complete a fused lactam ring of 4-7 members;
R³ represents aryl, heteroaryl, C₁₋₆alkyl, polyfluoro C₁₋₆alkyl, C₃₋₈cycloalkyl or C₃₋₈cycloalkylC₁₋₆alkyl;
where each R⁶ is independently selected from H, a polyfluoroC₁₋₆alkyl, or a C₁₋₆alkyl which is optionally substituted with halogen, —CN, —NO₂, —OH, —SH, —NH₂, a phenyl, a C₁₋₄alkoxy, a C₁₋₄alkylthio, a C₁₋₄alkylamino, a di(C₁₋₄alkyl)amino, —CO₂H, —CO₂C₁₋₄alkyl, —CONH₂, —CONHC₁₋₄alkyl and —CON(C₁₋₄alkyl)₂; or two R⁶ groups attached to a single nitrogen atom may complete a heterocyclic ring of from 3 to 12 members including the said nitrogen, the remaining atoms being selected from C, N, O and S, and the ring optionally bearing up to 3 substituents independently selected from a C₁₋₆alkyl, a polyfluoroC₁₋₆alkyl, a C₂₋₇acyl, —OH and —CONH₂;
R⁷ represents R⁶ that is other than H;
or a pharmaceutically acceptable salt thereof.

In Formula I, the term “aryl” refers to a phenyl which is optionally fused to a 5-7 membered saturated or unsaturated ring which may be carbocyclic or may comprise up to 3 heteroatoms selected from nitrogen, oxygen and sulphur, and which may be oxo-substituted, said phenyl and optional fused ring together bearing 0, 1, 2 or 3 substituents independently selected from a C₁₋₆alkyl [which is optionally substituted with halogen, —CN, —NO₂, —OH, —SH, —NH₂, a C₁₋₄alkoxy, a C₁₋₄alkylthio, a C₁₋₄alkylamino, a di(C₁₋₄alkyl)amino, —CO₂H, —CO₂C₁₋₄alkyl, —CONH₂, —CONHC₁₋₄alkyl or —CON(C₁₋₄alkyl)₂], a polyfluoroC₁₋₆alkyl, a halogen, —CN, —NO₂, a heteroaryl, —COR⁶, —CO₂R⁶, —CON(R⁶)₂, —OCOR⁷, —NR⁶COR⁷, —NR⁶SO₂R⁷, —SO₃R⁶, —SO₂N(R⁶)₂, —OR⁶, —SR⁶ or —N(R⁶)₂.

In Formula I, the term “heteroaryl” refers to a heteroaromatic ring of 5 or 6 members, at least one member being nitrogen, oxygen or sulphur and the remainder carbon, said ring optionally being fused to a 5, 6 or 7 membered saturated or unsaturated ring which may be carbocyclic or may comprise up to 3 heteroatoms selected from nitrogen, oxygen and sulphur, and which may be oxo-substituted heteroaromatic ring and optional fused ring together bearing 0, 1, 2 or 3 substituents independently selected from a C₁₋₆alkyl [which is optionally substituted with halogen, —CN, —NO₂, —OH, —SH, —NH₂, a C₁₋₄alkoxy, a C₁₋₄alkylthio, a C₁₋₄alkylamino, a di(C₁₋₄alkyl)amino, —CO₂H, —CO₂C₁₋₄alkyl, —CONH₂, —CONHC₁₋₄alkyl or —CON(C₁₋₄alkyl)₂], a polyfluoroC₁₋₆alkyl, a halogen, —CN, —NO₂, a phenyl, —COR^{E}, —CO₂R⁶, —CON(R⁶)₂, —OCOR⁷, —NR⁶COR⁷, —NR⁶SO₂R⁷, —SO₃R⁶, —SO₂N(R⁶)₂, —OR⁶, SR⁶ and —N(R⁶)₂

As another example, in some embodiments, a suitable GSI is a compound as disclosed in U.S. Pat. No. 6,984,626, e.g., a compound of Formula II:

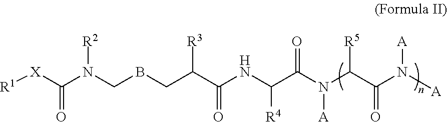

where R¹ is selected from:

(1) a C₁₋₁₀alkyl, a C₂₋₁₀alkenyl or a C₂₋₁₀alkynyl optionally substituted with 1, 2, or 3 substituents independently selected from:

(i) hydroxy;

(ii) carboxy;

(iii) a halogen;

(iv) a C₁₋₄alkoxy;

(v) a C₁₋₄alkoxycarbonyl;
(vi) —NR⁶R⁷, where R⁶ and R⁷ are independently selected from hydrogen, a C₁₋₅alkyl and C₁₋₅alkoxy C₁₋₅alkyl;
(vii) —CONR⁶R⁷ or OCONR⁶R⁷, where R⁶ and R⁷ are independently selected as defined above;
(viii) —N(R⁸)QR⁹, where Q is C(O), C(S), SO₂ or C(NH), R⁸ is hydrogen or a C₁₋₄alkyl, and R⁹ is hydrogen, a C₁₋₄alkyl, a C₁₋₄alkoxy, an amino, a C₁₋₄alkylamino, a di(C₁₋₄alkyl)amino, where each alkyl group is independently chosen;

(ix) a C₃₋₇cycloalkyl;

(x) a phenyl; a naphthyl; a five-membered heterocyclic ring containing 1, 2, 3 or 4 heteroatoms independently selected from O, N and S, at most one of the heteroatoms being O or S; or a six-membered heterocyclic ring containing 1, 2 or 3 nitrogen atoms; each of which is optionally substituted by one to three groups independently chosen from:
(a) halogen, cyano and nitro;
(b) hydroxy;
(c) C₁₋₄alkyl, C₂₋₄alkenyl and C₂₋₄alkynyl;
(d) C₁₋₄alkoxy;
(e) NR⁶R⁷ where R⁶ and R⁷ are independently selected as defined above;
(f) CO₂R⁸ where R⁸ is independently as defined above;
(g) CONR⁶R⁷ or OCONR⁶R⁷ wherein R⁶ and R⁷ are independently selected as defined above;
(h) SO₂NR⁶R⁷ where R⁶ and R⁷ are independently selected as defined above;
(i) CH₂NR⁶R⁷ where R⁶ and R⁷ are independently selected as defined above;
(j) N(R⁸)COR^{8′} where R⁸ is independently selected as defined above, and R^{8′} is independently selected as defined for R⁸; and
(k) NR⁸SO₂R^{8′} where R⁸ and R^{8′} are independently selected as defined above; or
   (2) phenyl or naphthyl; a five-membered heterocyclic ring containing 1, 2, 3 or 4 heteroatoms independently chosen from O, N and S, at most one of the heteroatoms being O or S; a six-membered heterocyclic ring containing 1, 2 or 3 nitrogen atoms; each of which is optionally substituted by one to three groups independently chosen from:
(a) halogen, cyano and nitro;
(b) hydroxy;
(c) a C₁₋₄alkyl, a C₂₋₄alkenyl and a C₂₋₄alkynyl;
(d) a C₁₋₄alkoxy;
(e) NR⁶R⁷ where R⁶ and R⁷ are independently selected as defined above;
(f) CO₂R⁸ where R⁸ is independently selected as defined above;
(g) CONR⁶R⁷ or OCONR⁶R⁷ wherein R⁶ and R⁷ are independently selected as defined above;
(h) SO₂NR⁶R⁷ wherein R⁶ and R⁷ are independently selected as defined above;
(i) CH₂NR⁶R⁷ wherein R⁶ and R⁷ are independently selected as defined above;
(j) N(R⁸)COR^{8′} wherein R⁸ and R^{8′} are independently selected as defined above; and
(k) NR⁸SO₂R⁸′ wherein R⁸ and R^{8′} are independently selected as defined above;
   where R² and R³ are independently chosen from a C₁₋₁₀alkyl, a C₁₋₁₀alkoxy, a C₂₋₁₀alkenyl, a C₂₋₁₀alkenyloxy, a C₂₋₁₀alkynyl or a C₂₋₁₀alkynyloxy; a phenyl; a naphthyl; a five-membered heteroaromatic ring containing 1, 2, 3 or 4 heteroatoms independently chosen from O, N and S, at most one of the heteroatoms being O or S; a six-membered heteroaromatic ring containing 1, 2 or 3 nitrogen atoms; and a group (CH₂)ₚQ¹ wherein Q¹ is a phenyl, a naphthyl, a five-membered heteroaromatic ring containing 1, 2, 3 or 4 heteroatoms independently selected from O, N and S, at most one of the heteroatoms being O or S, and a six-membered heteroaromatic ring containing 1, 2 or 3 nitrogen atoms; and where each of R² and R³ is independently optionally substituted by one to three groups independently selected from:
(a) halogen, cyano and nitro;
(b) hydroxy;
(c) a C₁₋₃alkyl, a C₂₋₃alkenyl and a C₂₋₃alkynyl;
(d) a C₁₋₃alkoxy;
(e) NR⁶R⁷ where R⁶ and R⁷ are independently selected as defined above;
(f) CO₂R⁸ where R⁸ is independently selected as defined above;
(g) CONR⁶R⁷ or OCONR⁶R⁷ where R⁶ and R⁷ are independently selected as defined above;
(h) SO₂NR⁶R⁷ wherein R⁶ and R⁷ are independently selected as defined above;
(i) CH₂NR⁶R⁷ where R⁶ and R⁷ are independently selected as defined above;
(j) N(R⁸)COR^{8′} wherein R⁸ and R^{8′} are in independently selected as defined above, (k) NR⁸SO₂R^{8′} where R⁸ and R^{8′} are independently selected as defined above; alternatively R³ may be hydrogen;
   where R⁴ and R⁵ are independently selected from hydrogen, a C₁₋₆alkyl optionally substituted by a halogen, hydroxy, thiol, an amino, a C₁₋₄alkoxy, a C₁₋₄alkylthio, carboxy or a C₁₋₄alkoxycarbonyl, and (CH₂)_{q}Q² wherein Q² is a five-membered unsaturated heterocycle containing 1, 2, 3 or 4 heteroatoms optionally chosen from O, N, and S providing that not more than one heteroatom is O or S, a six-membered unsaturated heterocycle containing 1, 2 or 3 N atoms, a phenyl or a naphthyl, each of the foregoing rings being optionally substituted with one to three groups independently chosen from hydroxy, a C₁₋₄alkyl, a C₁₋₄alkoxy, thiol, a C₁₋₄alkylthio, a halogen, an amino, carboxy, an amido, CO₂H and —NHC(NH₂)₂ and wherein each of the foregoing rings is optionally fused to a benzene ring; and
   where each A is independently selected from:
   (1) hydrogen;
   (2) a C₁₋₁₀alkyl, a C₂₋₁₀alkenyl or a C₂₋₁₀alkynyl optionally substituted with one to three substituents independently chosen from:
(i) hydroxy;
(ii) carboxy;
(iii) a halogen;
(iv) a C₁₋₄alkoxy;
(v) a C₁₋₄alkoxycarbonyl;
(vi) —NR⁶R⁷ wherein R⁶ and R⁷ are independently selected from hydrogen, C₁₋₅alkyl and C₁₋₅alkoxy C₁₋₅alkyl;
(vii) —CONR⁶R⁷ or OCONR⁶R⁷ wherein R⁶ and R⁷ are independently selected as defined above;
(viii) —N(R⁸)QR⁹ where:
Q is C(O), C(S), SO₂ or C(NH);
R⁸ is hydrogen or a C₁₋₄alkyl; and
R⁹ is hydrogen, a C₁₋₄alkyl, a C₁₋₄alkoxy, an amino, a C₁₋₄alkylamino, a di(C₁₋₄alkyl)amino, where each alkyl group is independently selected;
(ix) a C₃₋₇cycloalkyl;
(x) a phenyl or a naphthyl; a five-membered heterocyclic ring containing 1, 2, 3 or 4 heteroatoms independently selected from O, N and S, at most one of the heteroatoms being O or S; a six-membered heterocyclic ring containing 1, 2 or 3 nitrogen atoms; each of which is optionally substituted by one to three groups independently selected from:
(a) a halogen, cyano and nitro;
(b) hydroxy;
(c) a C₁₋₄alkyl, a C₂₋₄alkenyl and a C₂₋₄alkynyl;
(d) a C₁₋₄alkoxy;
(e) NR⁶R⁷ where R⁶ and R⁷ are independently selected as defined above;
(f) CO₂R⁸ wherein R⁸ is independently as defined above;
(g) CONR⁶R⁷ or OCONR⁶R⁷ wherein R⁶ and R⁷ are independently selected as defined above;
(h) SO₂NR⁶R⁷ where R⁶ and R⁷ are independently selected as defined above;
(i) CH₂NR⁶R⁷ where R⁶ and R⁷ are independently selected as defined above;
(j) N(R⁸)COR^{8′} where R⁸ is independently selected as defined above and R^{8′} is independently selected as defined for R⁸; and
(k) NR⁸SO₂R^{8′} wherein R⁸ and R^{8′} are independently selected as defined above; and
   (3) a seven-membered heterocycle:
   having an otherwise unsubstituted carbon atom at the point of attachment to the rest of the compound of formula I,
   having at a first atom alpha to the point of attachment a carbon atom which is unsubstituted or substituted by an oxygen or sulphur atom,
   having at a first atom beta to the point of attachment, which atom is alpha to the foregoing first atom alpha, a carbon atom or a nitrogen atom,
   having at a second atom alpha to the point of attachment a carbon atom, which is optionally substituted by oxygen, or a nitrogen atom,
   having at a second atom beta to the point of attachment, which atom is alpha to the foregoing second atom alpha, a carbon atom or a nitrogen atom,
   and having at the two remaining atoms carbon atoms;
   where the seven-membered heterocycle described above, a double bond may be present between the second atom alpha and the second atom beta;
   where the seven-membered heterocycle may be fused to one or two aromatic rings via any adjacent pair of atoms other than the point of attachment and the first atom alpha alone or in combination; the aromatic ring may be a benzene or a five-membered heterocycle containing 1, 2, 3 or 4 heteroatoms selected from O, N and S, providing that not more than one heteroatom is O or S or a six-membered heterocycle containing 1, 2 or 3 nitrogen atoms;
   alternatively, a pair of adjacent carbon atoms in the seven-membered heterocycle, other than the point of attachment and the first atom alpha alone or in combination may form part of a fused cyclopropyl or cyclopentyl ring;
   one to three substitutable atoms of the seven-membered heterocycle are optionally substituted by:
an aromatic ring as defined above optionally substituted by hydroxy, halogen, methoxy or alkyl having one to four carbon atoms;
an alkyl group having one to four carbon atoms optionally substituted by a halogen atom, hydroxy, an aromatic ring as defined above optionally substituted by hydroxy, halogen, methoxy or alkyl having one to four carbon atoms, cycloalkyl having three to seven carbon atoms, methoxy, bicycloalkyl having seven to twelve carbon atoms, heterocycle having five to seven atoms one of which is oxygen, nitrogen or sulphur which is optionally oxidized;
a heterocycle having five to seven atoms one of which is oxygen, nitrogen or sulphur which is optionally oxidized;
a cycloalkyl having three to seven carbon atoms; or
a bicycloalkyl having seven to twelve carbon atoms;
   or the two groups A attached to the same nitrogen atom, together with that atom, form: a five-membered heterocyclic ring optionally containing 1, 2 or 3 further heteroatoms chosen from O, N and S, not more than one of the heteroatoms being O or S; or a six-membered heterocyclic ring optionally containing 1 or 2 further nitrogen atoms; each of which is optionally substituted by one to three groups independently selected from:
(a) a halogen, cyano and nitro;
(b) hydroxy;
(c) a C₁₋₄alkyl, a C₂₋₄alkenyl and a C₂₋₄alkynyl;
(d) a C₁₋₄alkoxy;
(e) NR⁶R⁷ where R⁶ and R⁷ are independently selected as defined above;
(f) CO₂R⁸ where R⁸ is independently selected as defined above;
(g) CONR⁶R⁷ or OCONR⁶R⁷ where R⁶ and R⁷ are independently selected as defined above;
(h) SO₂NR⁶R⁷ where R⁶ and R⁷ are independently selected as defined above;
(i) CH₂NR⁶R⁷ where R⁶ and R⁷ are independently selected as defined above;
(j) N(R⁸)COR^{8′} where R⁸ is independently selected as defined above and R^{8′} is independently selected as defined for R⁸; and
(k) NR⁸SO₂R^{8′} where R⁸ and R^{8′} are independently selected as defined above;
   B is C═O or CHOH in the R configuration;
   X is oxygen or a bond;
   n is zero or one; and
   p is zero, one, two or three; and
   q is zero, one, two or three;
   with the proviso that no carbon atom is substituted by more than one hydroxy group.

As another example, in some embodiments, a suitable GSI is a compound as described in U.S. Pat. No. 6,756,511, e.g., a compound of Formula III:

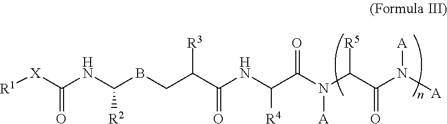

where R¹, X, R³, R⁴, A, R⁵ and n are as defined above for the compounds of Formula (II); R² is independently selected as described for R³.

As another example, in some embodiments, a suitable GSI is a cyclohexyl sulfone compound as described in U.S. Pat. No. 6,890,956, e.g., a compound of Formula IV:

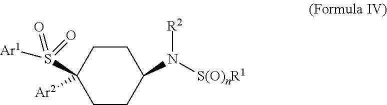

where n is 1 or 2;
R¹ is CF₃ or a C₁₋₆alkyl, a C₂₋₆alkenyl, a C₃₋₉cycloalkyl or a C₃₋₆cycloalkylC₁₋₆alkyl, any of which may bear up to 2 substituents selected from a halogen, CN, CF₃, OR³, COR³, CO₂R³, OCOR⁴, SO₂R⁴, N(R⁵)₂, and CON(R⁵)₂;
or R¹ is an aryl, an arylC₁₋₆alkyl, a C-heterocyclyl or a C-heterocyclylC₁₋₆alkyl;
R² is H or a C₁₋₄alkyl;
R³ is H, a C₁₋₄alkyl, a phenyl or a heteroaryl;
R⁴ is a C₁₋₄alkyl, a phenyl or a heteroaryl;
R⁵ is H or a C₁₋₄alkyl, or two R⁵ groups together with a nitrogen atom to which they are mutually attached complete an azetidine, pyrrolidine, piperidine, morpholine, thiomorpholine or thiomorpholine-1,1-dioxide ring;
Ar¹ and Ar² independently represent phenyl or heteroaryl, either of which bears 0, 1, 2 or 3 substituents independently selected from a halogen, CN, NO₂, CF₃, CHF₂, OH, OCF₃, CHO, CH═NOH, C₁₋₄alkoxy, C₁₋₄alkoxycarbonyl, C₂₋₆acyl, C₂₋₆alkenyl and C₁₋₄alkyl which optionally bears a substituent selected from halogen, CN, NO₂, CF₃, OH and C₁₋₄alkoxy;
where “aryl” at every occurrence thereof refers to phenyl or heteroaryl which optionally bear up to 3 substituents selected from halogen, CN, NO₂, CF₃, OCF₃, OR³, COR³, CO₂R³, OCOR⁴, N(R⁵)₂, CON(R⁵)₂ and optionally-substituted C₁₋₆alkyl, C₁₋₆alkoxy, C₂₋₆alkenyl or C₂₋₆alkenyloxy wherein the substituent is selected from halogen, CN, CF₃, phenyl, OR³, CO₂R³, OCOR⁴, N(R⁵)₂ and CON(R⁵)₂; and
where “C-heterocyclyl” and “N-heterocyclyl” at every occurrence thereof refer respectively to a heterocyclic ring system bonded through carbon or nitrogen, said ring system being non-aromatic and comprising up to 10 atoms, at least one of which is O, N or S, and optionally bearing up to 3 substituents selected from oxo, halogen, CN, NO₂, CF₃, OCF₃, OR³, COR³, CO₂R³, OCOR⁴, OSO₂R⁴, N(R⁵)₂, CON(R⁵)₂ and optionally-substituted phenyl, C₁₋₆alkyl, C₁₋₆alkoxy, C₂₋₆alkenyl or C₂₋₆alkenyloxy wherein the substituent is selected from halogen, CN, CF₃, OR³, CO₂R³, OCOR⁴, N(R⁵)₂ and CON(R⁵)₂;
or a pharmaceutically acceptable salt thereof.

As another example, in some embodiments, a suitable GSI is a compound as described in U.S. Publication No. 2005/0075320, e.g., a compound of Formula IV, where the —N(R²)S(O)ₙR¹ group shown in the structure is replaced with the following group:

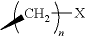

and where the Ar¹ group shown in the structure is replaced with —N(R⁶)₂;

where n is 0, 1, 2 or 3; and

X is H, a halogen, CN, N₃, OH, OR¹, N(R²)₂, CO₂H, CO₂R¹, OCOR¹, CHO, COR¹, CON(R²)₂, OCON(R²)₂, SCN, SR¹, S(O)R¹, SO₂R¹, SO₂N(R²)₂, OSO₂N(R²)₂, NHCOR¹, NHCO₂R¹, NHCON(R²)₂, NHSO₂R¹ or NHSO₂N(R²)₂;

each R⁶ is independently selected from H, a C₁₋₆alkyl, a C₃₋₆cycloalkyl and a C₂₋₆alkenyl, any of which is optionally substituted with up to 3 halogen atoms or with CN; or the two R⁶ groups and the nitrogen to which they are attached complete an N-heterocyclyl group or a heteroaryl group which is attached through N.

As another example, in some embodiments, a suitable GSI is a compound as described in PCT Publication No. WO0318543, e.g., a compound of Formula IV, where the —N(R²)S(O)ₙR¹ group shown in the structure is replaced with the following group:

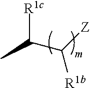

where m is 0 or 1;

Z is CN, an alkoxy, an alkenyloxy, an aryloxy, a carboxy alkyl, aryl or alkenyl ester, or a carboxy alkyl, aryl or alkenyl amide;

R^{1c} and R^{1b} are independently selected from hydrogen, a C₁₋₄alkyl and hydroxy.

As another example, in some embodiments, a suitable GSI is a compound as described in U.S. Pat. No. 6,683,091, e.g., a compound of Formula V or a pharmaceutically acceptable salt or solvate thereof:

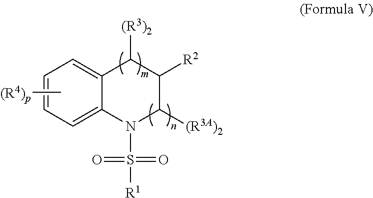

where R¹ is selected from an unsubstituted aryl, an aryl substituted with one or more (e.g., 1, 2 or 3) R⁵ groups, a heteroaryl, and a heteroaryl substituted with one or more (e.g., 1, 2 or 3) R⁵ groups;
R² is selected from an alkyl, —X(CO)Y, —(CR³)₁₋₄X(CO)Y; and any of the groups for R¹;
each R³ is independently selected from H and an alkyl;
each R^{3A} is independently selected from H and an alkyl;
R⁴ is independently selected from a halogen, —CF₃, —OH, —Oalkyl, —OCF₃, —CN, —NH₂, —CO₂alkyl, —CONR⁶R⁷, -alkylene-NR⁶R⁷, —NR⁶COalkyl, —NR⁶COaryl, —NR⁶COheteroaryl, and —NR⁶CONR⁶R⁷;
R⁵ is independently selected from a halogen, —CF₃, —OH, —Oalkyl, —OCF₃, —CN, —NH₂, —CO₂alkyl, —CONR⁶R⁷, an alkylene-NR⁶R⁷, —NR⁶COalkyl, —NR⁶COaryl, —NR⁶COheteroaryl, —NR⁶CONR⁶R⁷;
X is selected from —O—, —NH— and —N(alkyl)—;
Y is selected from —NR⁶R⁷ and —N(R³)(CH₂)₂₋₆NR⁶R⁷;
R⁶ and R⁷ are independently selected from H, an alkyl, a cycloalkyl, an arylalkyl, a heteroarylalkyl,

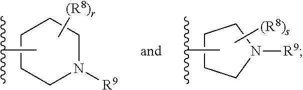

R⁶ and R⁷ taken together with the nitrogen atom to which they are bound form a heterocycloalkyl group selected from:

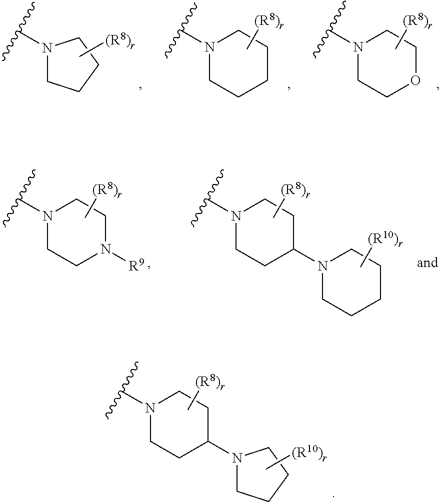

where each R⁸ is independently an alkyl optionally substituted with 1, 2, 3 or 4 hydroxy groups;
each R⁹ is independently selected from H, an alkyl, an alkyl substituted with 1, 2, 3 or 4 hydroxy groups, a cycloalkyl, a cycloalkyl substituted with 1, 2, 3 or 4 hydroxy groups, an arylalkyl, a heteroarylalkyl, a —COOalkyl, and any of the groups for R¹;
each R¹⁰ is independently selected from H, and an alkyl;
m is 0, 1, 2 or 3, and n is 0, 1, 2 or 3, such that m+n is 1, 2, 3 or 4;
p is 0, 1, 2, 3 or 4;
r is 0, 1, 2, 3 or 4;
s is 0, 1, 2 or 3; and

In certain embodiments, compounds of Formula V do not include:

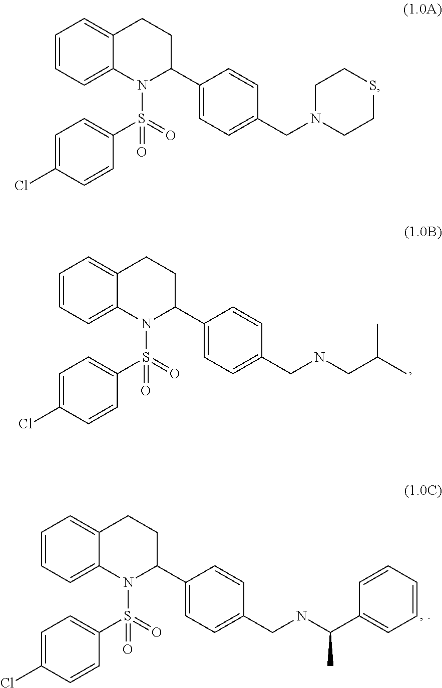

In certain embodiments, in compounds of Formula V when n=0 and m=2, R² is not an alkyl, a dialkyl or an alkenyl.

As another example, in some embodiments, a suitable GSI is a compound as described in U.S. Pat. No. 6,448,229, e.g., a compound of Formula VI or a pharmaceutically acceptable salt thereof:

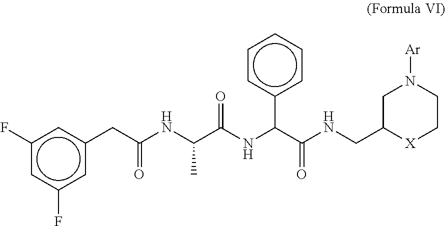

where X is CH₂, oxygen or sulphur; and

Ar is a phenyl optionally substituted with one, two or three substituents selected from a halogen, a C₁₋₆alkyl, a C₂₋₆alkenyl, a C₂₋₆alkynyl, hydroxy, cyano, nitro, NR₁R², where R¹ and R² are independently selected from hydrogen, a C1-6alkyl, a C1-6alkoxy, a C2-6alkenyloxy, a C2-6alkynyloxy, thiol, a C1-6alkylthio, a C₂₋₆alkenylthio, a C₂₋₆alkynylthio, a C₁₋₆alkylcarbonyl, a C₁₋₆alkoxycarbonyl, a C₁₋₆haloalkyl, a C₂₋₆haloalkenyl and a C₂₋₆haloalkynyl.

As another example, in some embodiments, a suitable GSI is a compound as described in U.S. Pat. No. 5,703,129, e.g., a compound of Formula VII or a pharmaceutically acceptable salt or hydrates thereof:

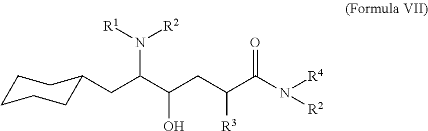

where R¹ is selected from a C₄₋₈alkyl, a C₄₋₈alkenyl, a C₁₋₄alkoxy-C₁₋₄alkanediyl, a R⁵-substituted C₃₋₆cycloalkyl, a R⁵-substituted C₃₋₆cycloalkyl-lower-alkanediyl, and Ar—(CH₂)ₙ— in which Ar is selected from

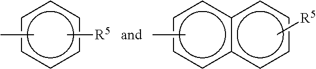

where R⁵ is hydrogen, a lower (C1-6) alkyl, or lower alkoxy, and n is 1, 2, 3 or 4;

each R² is independently selected from hydrogen and methyl;

R³ is selected from a lower alkyl, a C₃₋₆cycloalkyl, a C₃₋₆cycloalkyl-lower-alkanediyl, a C₃₋₆alkenyl, and Ar—(CH₂)ₙ—; and

R⁴ is selected from R³, a lower alkyl-thio-lower alkyl, and

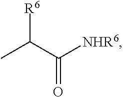

where R⁶ is lower alkyl.

As another example, in some embodiments, a suitable GSI is a compound as described in U.S. Publication No. 2009/0118289, e.g., a compound of Formula VIII or a pharmaceutically acceptable salt thereof:

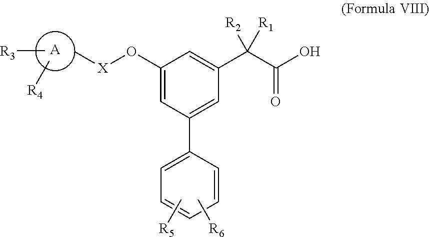

where A is a ring selected from the group consisting of a phenyl, a C₃₋₇cycloalkyl and a heterocyclyl;

X is a linear C₁-C₄ alkylene group which is optionally substituted with one or more substituents selected from the group consisting of F, Cl, Br, I and C₁-C₄ alkyl, wherein the C₁-C₄ alkyl group is optionally be substituted with one or more substituents selected from the group consisting of F, Cl, Br, and I;

R1 and R2 are each independently selected from the group consisting of H; an alkyl selected from the group consisting of CH₃, C₂H₅, i-C₃H₇, n-C₃H₇, i-C₄H₉, n-C₄H₉, sec-C₄H₉, and tert-C₄H₉; and alkenyl selected from the group consisting of C₂H₃, i-C₃H₅, n-C₃H₅, n-C₄H₇, i-C₄H₇, and sec-C₄H₇; or
R¹ and R² together form a ring, either saturated or unsaturated, with the carbon atom to which they are attached having 3-6 carbon atoms, which may contain in the ring one or more heteroatoms from the group N, S or O, wherein the heteroatoms may be identical or different if more than one heteroatom is present;
R³, R⁴, R⁵ and R⁶ are independently selected from the group consisting of H, F, Cl, Br, I, CN, OH, C(O)N(R⁷R⁸), S(O)₂R⁷, SO₂N(R⁷R⁸), S(O)N(R⁷R⁸), N(R⁷)S(O)₂R⁸, N(R⁸)S(O)R⁸, S(O)₂R⁷, N(R⁷)S(O)₂N(R⁸R^{8a}), SR⁷, N(R⁷R⁸), N(R⁷)C(O)R⁸, N(R⁷)C(O)N(R⁸R^{8a}), N(R⁷)C(O)OR⁸, OC(O)N(R⁷R⁸), C(O)R⁷, a substituted or unsubstituted C₁-C₄-alkyl, and a substituted or unsubstituted C₁-C₄-alkoxy; where the substituents of the C₁-C₄-alkyl and C₁-C₄-alkoxy groups are selected from the group consisting of F, Cl, Br, I, and CF₃; and
R⁷, R⁸ and R^{8a} are independently selected from the group consisting of H, a C₁-C₄-alkyl, a heterocyclyl; and a C₃₋₇cycloalkyl, where the C₁-C₄-alkyl, heterocyclyl, and C₃₋₇cycloalkyl groups are optionally substituted with one or more substituents selected from the group consisting of F, Cl, Br, I and CF₃.

As another example, in some embodiments, a suitable GSI is a compound as described in U.S. Publication No. 2009/0105345, e.g., a compound of Formula IX:

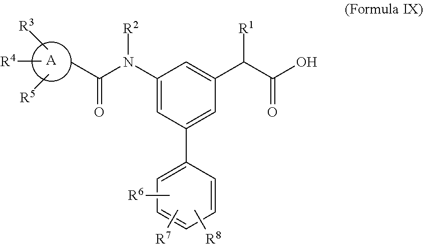

where A is selected from the group consisting of a phenyl, a heterocyclyl, and a heteroaryl;

R¹ is selected from the group consisting of H; an alkyl selected from the group consisting of CH₃, C₂H₅, i-C₃H₇, n-C₃H₇, i-C₄H₉, n-C₄H₉, sec-C₄H₉, and tert-C₄H₉; and an alkenyl selected from the group consisting of C₂H₃, i-C₃H₅, n-C₃H₅, n-C₄H₇, i-C₄H₇, and sec-C₄H₇; where the alkyl and alkenyl groups are optionally substituted with one, two, or three substituents independently selected from the group consisting of F, Cl, Br, I and CF₃;

R² is selected from the group consisting of H, benzyl; alkyl selected from the group CH₃, C₂H₅, i-C₃H₇, n-C₃H₇, i-C₄H₉, n-C₄H₉, sec-C₄H₉, and tert-C₄H₉; CH₂CH₂CH(CH₃)₂ and alkenyl selected from C₂H₃, i-C₃H₅, n-C₃H₅, n-C₄H₇, i-C₄H₇, sec-C₄H₇; wherein the alkyl and alkenyl groups are optionally substituted with one, two, or three substituents independently selected from the group consisting of F, Cl, Br, I, and CF₃;

R³ and R⁶, are independently selected from the group consisting of H, F, Cl, Br, I, CN, OH, C(O)N(C₁₋₄alkyl)₂, S(O)₂C₁₋₄alkyl, SO₂N(C₁₋₄alkyl)₂, S(O)N(C₁₋₄alkyl)₂, N(C₁₋₄alkyl)S(O)₂C₁₋₄alkyl, N(C₁₋₄alkyl)S(O)C₁₋₄alkyl, S(O)₂C₁₋₄alkyl, N(C₁₋₄alkyl)S(O)₂N(C₁₋₄alkyl)₂, SC₁₋₄alkyl, N(C₁₋₄alkyl)₂, N(C₁₋₄alkyl)C(O)C₁₋₄alkyl, N(C₁₋₄alkyl)C(O)N(C₁₋₄alkyl)₂, N(C₁₋₄alkyl)C(O)OC₁₋₄alkyl, OC(O)N(C₁₋₄alkyl)₂, C(O)C₁₋₄alkyl, substituted and unsubstituted C₁-C₄-alkyl and substituted and unsubstituted C₁-C₄-alkoxy, and wherein the substituents of both groups C₁-C₄-alkyl and C₁-C₄-alkoxy are selected from F, Cl, Br, I, CF₃;

R⁴, R⁵, R⁷ and R⁸ are independently selected from the group consisting of OCF₃, CF₃, H, F, Cl, OCH₃, C₁₋₄alkyl, and CN; and

solvates, hydrates, esters, and pharmaceutically acceptable salts thereof.

As another example, in some embodiments, a suitable GSI is a compound as described in U.S. Publication No. 2009/0105275, e.g., a compound of Formula X:

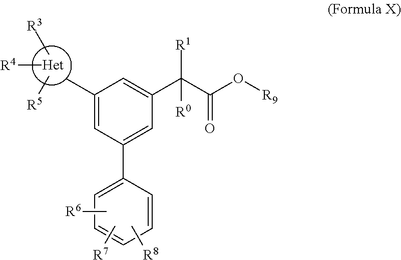

where Het is:

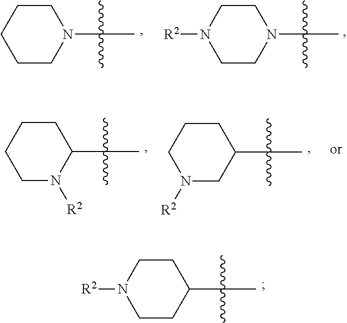

R⁰ is H or F;

R² is selected from the group consisting of H, a cyclohexyl,

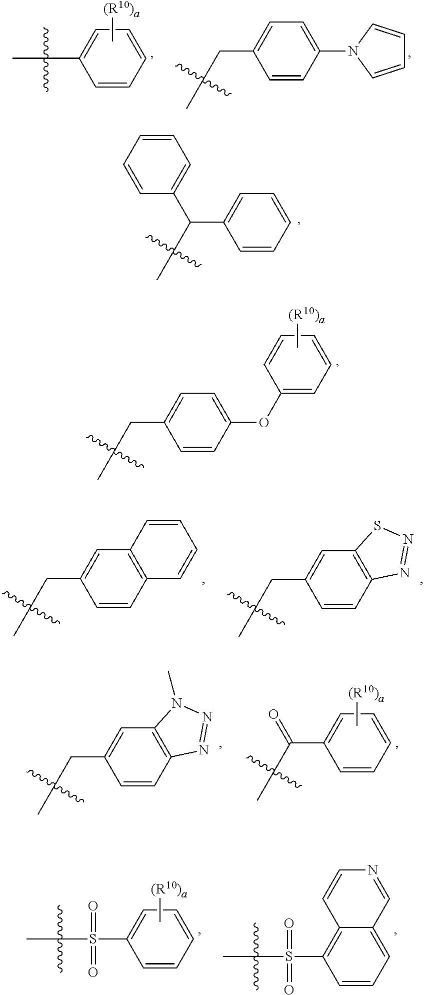

SO₂CH₃, alkyl selected from the group consisting of CH₃, C₂H₅, i-C₃H₇, n-C₃H₇, i-C₄H₉, n-C₄H₉, sec-C₄H₉, tert-C₄H₉, CH₂CH₂CH(CH₃)₂, CH₂CH₂CH₂CH(CH₃)₂, CH₂CH₂C(CH₃)₃, CH(CH₂CH₃)₂, and C(O)CH₂CH(CH₃)₂; alkenyl selected from the group consisting of C₂H₃, i-C₃H₅, n-C₃H₅, n-C₄H₇, i-C₄H₇, sec-C₄H₇, and CH₂CH═CHCH(CH₃)₂; where the alkyl and alkenyl groups are optionally substituted with F, Cl, Br, I, CF₃, -heteroaryl-(R¹⁰)ₙ, or

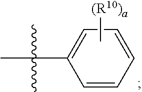

where R¹⁰ is CF₃, OCF₃, H, F, Cl, OCH₃, C₁₋₄alkyl, or CN; and n is 1, 2, or 3; alternatively, R² can be two C₁₋₄alkyl groups, so that their attached nitrogen is quaternized;

R⁹ is selected from the group consisting of H, alkyl selected from the group CH₃, C₂H₅, i-C₃H₇, n-C₃H₇, i-C₄H₉, n-C₄H₉, sec-C₄H₉, tert-C₄H₉; alkenyl selected from C₂H₃, i-C₃H₅, n-C₃H₅, n-C₄H₇, i-C₄H₇, sec-C₄H₇; wherein said alkyl and alkenyl groups are optionally substituted with one, two, or three substituents independently selected from the group consisting of F, Cl, Br, I and CF₃; and

R¹, R³, R⁴, R⁵, R⁶, R⁷ and R⁸ are as described above for Formula IX; and

solvates, hydrates, esters, and pharmaceutically acceptable salts thereof.

As another example, in some embodiments, a suitable GSI is a compound as described in U.S. Publication No. 2008/0058316, e.g., a compound of Formula XI:

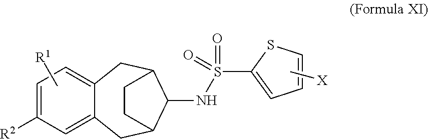

where X is a halogen;

R¹ is hydrogen, a halogen, hydroxy, a C₁₋₆alkyl or a C₁₋₄alkoxy;

R² is a radical of the following structure:

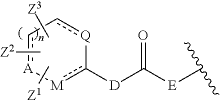

where E is CH₂ or NH;

D is (CH₂)ₘ, O(CH₂)ₘ, HN(CH₂)ₙ, or CH═CH; where m is 0, 1 or 2; A and Q are independently N, NCH₃ or C;

M is C or C═O;

n is 1 or 2;

Z¹ and Z² are independently H, halo, halo(C₁₋₄)alkyl, phenyl, or Z¹ and Z², when attached to carbon atoms, form a 6-membered aryl ring with the carbon atoms to which they are attached; and

Z³ is H, halo, halo(C₁₋₄)alkyl or phenyl.

As another example, in some embodiments, a suitable GSI is a compound as described in U.S. Publication No. 2005/0143369, e.g., a compound of Formula XII:

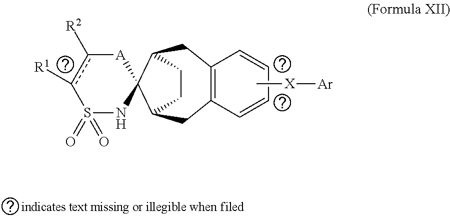

where the moiety X—Ar is attached at one of the positions indicated by an asterisk;

X is a bivalent residue of a heteroaryl ring comprising 5 ring atoms of which two or three are selected from O, N and S, optionally bearing a hydrocarbon substituent comprising 1-5 carbon atoms which is optionally substituted with up to 3 halogen atoms;

Ar is phenyl or 6-membered heteroaryl, either of which bears 0, 1, 2 or 3 substituents independently selected from halogen, CF₃, CHF₂, CH₂F, NO₂, CN, OCF₃, C₁₋₆alkyl and C₁₋₆alkoxy; A is (CH₂)ₙ where n is 0, 1 or 2;

bond a is single or double;

R¹ is H or C₁₋₆alkyl, C₂₋₆alkenyl, or C₂₋₆alkynyl, any of which optionally is substituted with up to 5 fluorine atoms; or R¹ and R² together complete a fused benzene ring which is optionally substituted with up to 3 halogen atoms or C₁₋₄alkyl groups; and

R² is H or together with R¹ completes a fused benzene ring as described above;

or a pharmaceutically acceptable salt thereof.

As another example, in some embodiments, a suitable GSI is a compound as described in U.S. Publication No. 2005/0261276, e.g., a compound of Formula XIII:

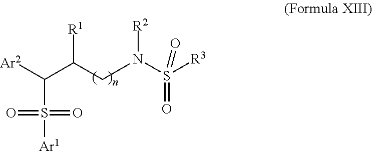

where n is 2, 3 or 4;

Ar¹ is a phenyl or a heteroaryl, either of which bears 0, 1, 2 or 3 substituents independently selected from a halogen, CN, NO₂, CF₃, CHF₂, OH, OCF₃, a C₁₋₄alkoxy or a C₁₋₄alkyl which optionally bears a substituent selected from a halogen, CN, NO₂, CF₃, OH and a C₁₋₄alkoxy;

Ar² is a phenyl or a heteroaryl, either of which bears 0, 1, 2 or 3 substituents independently selected from halogen, CN, NO₂, CF₃, CHF₂, OH, OCF₃, a C₁₋₄alkoxy or a C₁₋₄alkyl which optionally bears a substituent selected from a halogen, CN, NO₂, CF₃, OH and a C₁₋₄alkoxy;

R¹ is a C₁₋₄alkyl, or together with R² completes a pyrrolidine, a piperidine or a homopiperidine ring;

R² is H or a C₁₋₆alkyl which optionally bears a substituent selected from a halogen, CN, NO₂, CF₃, OH and a C₁₋₄alkoxy; or together with R¹ completes a pyrrolidine, piperidine or homopiperidine ring; or together with R³ completes a tetrahydroisothiazole-1,1-dioxide ring; and

R³ is a phenyl, a naphthyl or a heteroaryl, any of which may bear up to 3 substituents selected from halogen, CN, NO₂, CF₃, CHF₂, OH, OCF₃, a C₁₋₄alkoxy, a C₁₋₄alkoxycarbonyl, a C₂₋₆acyl, a C₂₋₆acyloxy, a C₂₋₆acylamino, an amino, a C₁₋₄alkylamino, a di(C₁₋₄alkyl)amino or a C₁₋₄alkyl which optionally bears a substituent selected from a halogen, CN, NO₂, CF₃, OH and C₁₋₄alkoxy; or R³ represents CF₃ or a non-aromatic hydrocarbon group of up to 6 carbon atoms optionally bearing one substituent selected from a halogen, CN, CF₃, OH, OCF₃, a C₁₋₄alkoxy, a C₁₋₄alkoxycarbonyl, a C₂₋₆acyl, a C₂₋₆acyloxy, a C₂₋₆acylamino, an amino, a C₁₋₄alkylamino, a di(C₁₋₄alkyl)amino, a phenyl, a naphthyl or a heteroaryl, any of which may bear up to 3 substituents selected from a halogen, CN, NO₂, CF₃, CHF₂, OH, OCF₃, a C₁₋₄alkoxy, a C₁₋₄alkoxycarbonyl, a C₂₋₆acyl, a C₂₋₆acyloxy, a C₂₋₆acylamino, an amino, a C₁₋₄alkylamino, a di(C₁₋₄alkyl)amino or a C₁₋₄alkyl which optionally bears a substituent selected from a halogen, CN, NO₂, CF₃, OH and a C₁₋₄alkoxy; or R³ together with R² completes a tetrahydroisothiazole-1,1-dioxide ring;

or a pharmaceutically acceptable salt thereof.

As another example, in some embodiments, a suitable GSI is a compound as described in U.S. Publication No. 2006/0009467, e.g., a compound of Formula XIV:

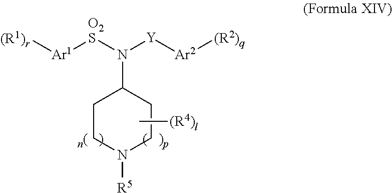

where Ar¹ and Ar² are independently selected from aryl or heteroaryl;

Y is a bond, or Y is a —(C(R³)₂)₁₋₃— group;

each R¹ is independently selected from —(C₁-C₆)alkyl, an aryl; an aryl substituted with one or more substituents independently selected from a halogen, CF₃, a (C₁-C₆)alkyl, a (C₁-C₆)alkoxy, OCF₃, NH₂, or CN; a heteroaryl; a heteroaryl substituted with one or more substituents independently selected from a halogen, CF₃, a (C₁-C₆)alkyl, a (C₁-C₆)alkoxy, OCF₃, NH₂, or CN; a halogen, —CF₃, —OCF₃, —CN, —NO₂, —NH₂, —C(O)NH(C₁-C₆)alkyl, —C(O)N((C₁-C₆)alkyl)₂ (wherein each (C₁-C₆)alkyl group is the same or different), —C(O)N((C₁-C₆)alkyl)₂ (wherein each (C₁-C₆)alkyl group is the same or different, and the (C₁-C₆)alkyl groups taken together with the nitrogen to which they are bound form a ring), —NHC(O)(C₁-C₆)alkyl, —NHC(O)O(C₁-C₆)alkyl, —NHC(O)NH(C₁-C₆)alkyl, —NHSO₂(C1-C₆)alkyl, —OH, —OC(O)(C₁-C₆)alkyl, —O(C₁-C₆)alkyl, —Oaryl and —Oaryl(C₁-C₆)alkyl;

each R² is independently selected from a —(C₁-C₆)alkyl, —CF₃, —OCF₃, —CN, —NO₂, —NH₂, —C(O)O(C₁-C₆)alkyl, —C(O)NH(C₁-C₆)alkyl, —N((C₁-C₆)alkyl)₂ (wherein each (C₁-C₆)alkyl group is the same or different), —N((C₁-C₆)alkyl)₂ (wherein each (C₁-C₆)alkyl group is the same or different, and the (C₁-C₆)alkyl groups taken together with the nitrogen to which they are bound form a ring), —NHC(O)(C₁-C₆)alkyl, —NHC(O)O(C₁-C₆)alkyl, —NHC(O)NH(C₁-C₆)alkyl, —NHSO₂(C₁-C₆)alkyl, —OH, —OC(O)(C₁-C₆)alkyl, —O(C₁-C₆)alkyl, —Oaryl, —Oaryl(C₁-C₆)alkyl, an aryl; an aryl substituted with one or more substituents independently selected from a halogen, CF₃, (C₁-C₆)alkyl, (C₁-C₆)alkoxy, OCF₃, NH₂, or CN; a heteroaryl; a heteroaryl substituted with one or more substituents independently selected from a halogen, CF₃, (C₁-C₆)alkyl, (C₁-C₆)alkoxy, OCF₃, NH₂, or CN; one of a group selected from:

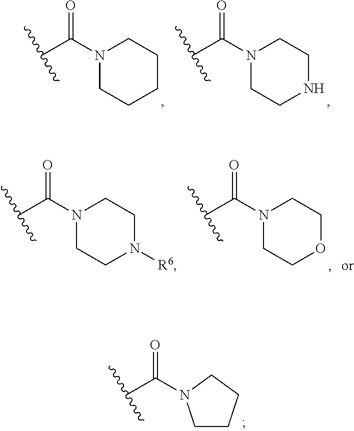

—C(O)N((C₁-C₆)alkyl)₂ (where each alkyl group is independently selected), and —C(O)N((C₁-C₆)alkyl)₂ (where each alkyl group is independently selected and wherein the alkyl groups taken together with the nitrogen atom form a heterocycloalkyl ring);

each R³ is independently selected from H and a (C1-C3)alkyl;

each R⁴ is independently selected from a (C₁-C₃)alkyl, OH and a —O(C₁-C₃)alkyl;

R⁵ is selected from hydrogen, a (C₁-C₆)alkyl, an aryl, a heteroaryl, a (C₁-C₃)alkylene-O(C₁-C₃)alkyl, a (C₁-C₆)alkylene-S(O)₀₋₂(C₁-C₃)alkyl, a (C₁-C₆)alkylene-S(O)₀₋₂NH(C₁-C₃)alkyl, a —C(O)(C₁-C₆)alkyl, a —C(O)aryl, a —C(O)aryl(C₁-C₃)alkyl, a —C(O)heteroaryl, a —C(O)heteroar(C₁-C₃)alkyl, a —C(O)O(C₁-C₆)alkyl, a —C(O)NH(C₁-C₆)alkyl, a —C(O)N((C₁-C₆)alkyl)₂ (where each C₁-C₆alkyl group is the same or different), a —C(O)N((C₁-C₆)alkyl)₂ (where each C₁-C₆alkyl group is the same or different and wherein the C₁-C₆ alkyl groups taken together with the nitrogen to which they are bound form a heterocycloalkyl ring), a —C(O)(C₁-C₃)alkylene-NH(C₁-C₃)alkyl, a —C(O)(C₁-C₃)alkylene-N((C₁-C₃)alkyl)₂ wherein each alkyl group is independently selected, a —SO₂(C₁-C₆)alkyl, a —SO₂NH(C₁-C₆)alkyl, a —SO₂N((C₁-C₆)alkyl)₂ where each C₁-C₆alkyl is the same or different, a —SO₂N((C₁-C₆)alkyl)₂ where each C₁-C₆alkyl is the same or different, and where the C₁-C₆ alkyl groups taken together with the nitrogen to which they are bound form a heterocycloalkyl ring, and one of a group of the formula:

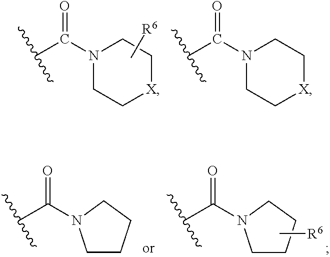

R⁶ is H or a (C₁-C₆)alkyl;

X is selected from CH₂, O, S, SO, SO₂, or N—R⁷;

where R⁷ is selected from a —(C₁-C₆)alkyl, a —(C₃-C₆)cycloalkyl, a —(C₁-C₃)alkylene-(C₃-C₆)cycloalkyl, an aryl, an ar(C₁-C₃)alkyl, a heteroaryl, a heteroar(C₁-C₃)alkyl, a —C(O)(C₁-C₆)alkyl, a —C(O)aryl, a —C(O)ar(C₁-C₃)alkyl, a —C(O)heteroaryl, a —C(O)heteroar(C₁-C₃)alkyl, a —C(O)O(C₁-C₆)alkyl, a —C(O)NH(C₁-C₆)alkyl, a —C(O)N((C₁-C₆)alkyl)₂ (where each C₁-C₆alkyl group is the same or different), a —C(O)N((C₁-C₆)alkyl)₂ (where each C₁-C₆alkyl group is the same or different, and the C₁-C₆alkyl groups taken together with the nitrogen to which they are bound form a heterocycloalkyl ring), a —C(O)(C₁-C₃)alkylene-NH(C₁-C₃)alkyl, a —C(O)(C₁-C₃)alkylene-N((C₁-C₃)alkyl)₂ (where the C₁-C₃alkyl groups are the same or different), and a —(C₁-C₃)alkylene-O—(C₁-C₃)alkyl;

n and p are independently selected from 0, 1, 2 and 3 to provide a 4 to 7 member ring;

r is 0, 1, 2 or 3;

q is 0, 1, 2 or 3; and

t is 0, 1, 2 or 3.

As another example, in some embodiments, a suitable GSI is a compound as described in U.S. Publication No. 2006/0009467, e.g., a compound of Formula XV:

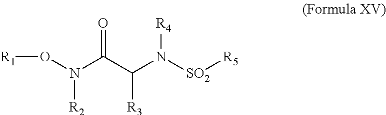

where R¹ is selected from hydrogen, an alkyl, an alkanoyl, an arylalkyl, and an arylalkanoyl, where the arylalkyl and arylalkanoyl groups are unsubstituted or substituted with 1, 2, 3, 4, or 5 R⁶ groups;

R⁶ at each occurrence is independently selected from a halogen, hydroxy, —NO₂, —CO₂R¹⁰, —CN, an alkyl, an alkoxy, a haloalkyl, and a haloalkoxy;

R² is selected from hydrogen, an alkyl, an alkoxy, an alkanoyl, an arylalkyl and an arylalkanoyl, where the arylalkyl and arylalkanoyl groups are unsubstituted or substituted with 1, 2, 3, 4, or 5 R⁶ groups;

R³ is —Z-Q-J, where
Z is selected from an alkyl, an alkoxyalkyl, an alkylthioalkyl, and an alkenyl, each of which is unsubstituted or substituted with 1 or 2 groups that are independently selected from an alkoxy, hydroxy, and a halogen;
Q is selected from a direct bond between Z and J, —C(═O)—, an aryl, a heteroaryl, and a heterocycloalkyl, where the aryl, heteroaryl, or heterocycloalkyl group is unsubstituted or substituted with 1 or 2 groups that are independently selected from an alkyl, a halogen, —NRBR⁹, and an alkoxy;

J is selected from —NR⁸R⁹, —NR⁷C(═O)NR⁸R⁹, —NR⁷C(═O)alkylNR⁸R⁹, —NR⁷C(═O)OR⁹, —C(═NR⁷)NR⁸R⁹, and —NH—C(═NR⁷)NR⁸R, where

R⁷ is selected from H, CN, NO₂, an alkyl, an alkanoyl, an arylalkanoyl and —C(═O)NR¹⁰R¹¹, where

R¹⁰ and R¹¹ are independently selected from H, and an alkyl, and

R⁸ and R⁹ are independently selected from H, an alkyl, hydroxy, an alkoxy, an alkoxyalkyl, a heterocycloalkylalkyl, an arylalkyl, and a heteroarylalkyl, where each of the above is unsubstituted or substituted with 1, 2, 3, or 4 R⁶ groups; or

R⁸ and R⁹ and the nitrogen to which they are attached form a 5, 6 or 7-membered heterocycloalkyl ring, which is unsubstituted or substituted with 1, 2, or 3 groups that are independently selected from an alkyl, an alkoxy, hydroxy, and a halogen; or

R⁷, R⁸ and the nitrogens to which they are attached form a 5, 6 or 7 membered heterocycloalkyl group that is unsubstituted or substituted with 1, 2 or 3 groups that are independently selected from alkyl, alkoxy, hydroxy, and halogen; and

R⁹ is selected from H, an alkyl, hydroxy, an alkoxy, an alkoxyalkyl, a heterocycloalkylalkyl, an arylalkyl, and a heteroarylalkyl, where each of the above is unsubstituted or substituted with 1, 2, 3, or 4 R⁶ groups;

R⁴ is selected from H, alkyl, and arylalkyl, wherein the arylalkyl group is unsubstituted or substituted with 1, 2, 3, 4, or 5 R⁶ groups; and

R⁵ is -M-G-A, where

M is selected from an aryl and a heteroaryl, where M is unsubstituted or substituted with 1, 2, 3, or 4 groups that are independently selected from a halogen, an alkyl, hydroxy, an alkoxy, a haloalkyl, —CN, a haloalkoxy, and a hydroxyalkyl;

G is selected from a direct bond between M and A, CH₂, -alkyl-O—, —Oalkyl-, O, S, SO, and SO₂;

A is selected from an aryl and a heteroaryl, where A is unsubstituted or substituted with 1, 2, 3, 4, or 5 groups that are independently selected from a halogen, an alkyl, an alkoxy, a haloalkyl, an aryloxy, a heteroaryloxy, an arylalkoxy, a heteroarylalkoxy, a haloalkoxy, —CN, and NO₂.

In some embodiments, in Formula XV, when M is phenyl, G is a direct bond between M and A, and A is phenyl, then at least one of the four remaining hydrogens on the phenyl ring of M, of M-G-A, must be substituted with a group independently selected from a halogen, an alkyl, hydroxy, an alkoxy, a haloalkyl, —CN, a haloalkoxy, and a hydroxyalkyl.

As another example, in some embodiments, a suitable GSI is a compound as described in PCT Publication No. WO01/70677, e.g., a sulphonamido-substituted bridged bicycloalkyl compound.

In some embodiments, the γ-secretase inhibitor selectively inhibits γ-secretase-mediated cleavage of a NICD polypeptide from the transmembrane domain of the Notch1 polypeptide.

### Screening Methods

The present disclosure provides methods of identifying agents that modulate Notch1/β-catenin binding, and methods of identifying agents that inhibit enzyme-mediated cleavage of Notch1 intracellular domain from Notch1 transmembrane domain.

Methods of Identifying Agents that Inhibit Binding of NICD to β-Catenin

The present disclosure provides an in vitro method for identifying an agent that blocks binding of an intracellular domain of a Notch1 polypeptide to β-catenin. The method generally involves: a) contacting a Notch1 polypeptide that comprises the intracellular domain of a Notch1 polypeptide with a test agent and a β-catenin polypeptide; and b) determining the effect, if any, of the test agent on binding of the Notch1 polypeptide to the β-catenin polypeptide. A test agent that reduces binding of the Notch1 polypeptide to the β-catenin polypeptide by at least about 10% is a candidate agent for increasing stem cell self-renewal and/or expansion.

The present disclosure provides an in vitro method of identifying an agent that increases binding of β-catenin to an intracellular domain of a Notch1 polypeptide. The method generally involves: a) contacting a Notch1 polypeptide that comprises the intracellular domain of a Notch1 polypeptide with a test agent and a β-catenin polypeptide; and b) determining the effect, if any, of the test agent on binding of the Notch1 polypeptide to the β-catenin polypeptide. A test agent that increases binding of β-catenin to an intracellular domain of a Notch1 polypeptide is a candidate agent for reducing cell proliferation.

A β-catenin polypeptide suitable for use in a subject screening method can comprise an amino acid sequence having at least about 85%, at least about 90%, at least about 95%, at least about 98%, at least about 99%, or 100%, amino acid sequence identity to a contiguous stretch of at least 25 amino acids, at least 50 aa, at least 100 aa, at least 200 aa, at least 300 aa, at least 400 aa, at least 500 aa, at least 600 aa, at least 700 aa, or 781 aa, of the amino acid sequence set forth in SEQ ID NO:3 and depicted in FIG. 9.

A subject screening method can be carried out in a cell-free assay.

A Notch1 polypeptide suitable for use in a subject screening method can comprise an amino acid sequence having at least about 85%, at least about 90%, at least about 95%, at least about 98%, at least about 99%, or 100%, amino acid sequence identity to a contiguous stretch of at least 25 amino acids, at least 50 aa, at least 100 aa, at least 200 aa, at least 300 aa, at least 400 aa, at least 500 aa, at least 600 aa, at least 700 aa, or 796 aa, of amino acids 1759-2556 of the amino acid sequence set forth in SEQ ID NO:1 and depicted in FIGS. 7A and 7B.

Determining an effect of a test agent on binding of a NICD to a β-catenin polypeptide can be carried out using, e.g., a protein blot assay, an enzyme-linked immunosorbent assay, a BRET assay, a FRET assay, or an immunoprecipitation assay.

By “test agent,” “candidate agent,” and grammatical equivalents herein, which terms are used interchangeably herein, is meant any molecule (e.g. proteins (which herein includes proteins, polypeptides, and peptides), small (i.e., 5-1000 Da, 100-750 Da, 200-500 Da, or less than 500 Da in size), or organic or inorganic molecules, polysaccharides, polynucleotides, etc.) which are to be tested for activity in inhibiting binding between a NICD polypeptide and a β-catenin polypeptide.

A variety of different candidate agents may be screened by the above methods. Candidate agents encompass numerous chemical classes, e.g., small organic compounds having a molecular weight of more than 50 daltons and less than about 10,000 daltons, less than about 5,000 daltons, or less than about 2,500 daltons. Candidate agents can comprise functional groups necessary for structural interaction with proteins, e.g., hydrogen bonding, and can include at least an amine, carbonyl, hydroxyl or carboxyl group, or at least two of the functional chemical groups. The candidate agents can comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof.

Candidate agents are obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs. Moreover, screening may be directed to known pharmacologically active compounds and chemical analogs thereof, or to new agents with unknown properties such as those created through rational drug design.

In one embodiment, candidate modulators are synthetic compounds. Any number of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

In another embodiment, the candidate modulators are provided as libraries of natural compounds in the form of bacterial, fungal, plant and animal extracts that are available or readily produced. Additionally, natural or synthetically produced libraries and compounds are readily modified through conventional chemical, physical and biochemical means. Known pharmacological agents may be subjected to directed or random chemical modifications, including enzymatic modifications, to produce structural analogs.

In one embodiment, candidate modulators include proteins (including antibodies, antibody fragments (i.e., a fragment containing an antigen-binding region, e.g., a FAb), single chain antibodies, and the like), nucleic acids, and chemical moieties. In one embodiment, the candidate modulators are naturally occurring proteins or fragments of naturally occurring proteins. Thus, for example, cellular extracts containing proteins, or random or directed digests of proteinaceous cellular extracts, may be tested, as is more fully described below. In this way libraries of prokaryotic and eukaryotic proteins may be made for screening against any number of ubiquitin ligase compositions. Other embodiments include libraries of bacterial, fungal, viral, and mammalian proteins.

In one embodiment, the candidate modulators are organic moieties. In this embodiment, as is generally described in WO 94/243 14, candidate agents are synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or candidate agents which can then be tested using the present invention.

As used herein, the term “determining” refers to both quantitative and qualitative determinations and as such, the term “determining” is used interchangeably herein with “assaying,” “measuring,” and the like.

Determining the effect, if any, of a test agent on binding between a NICD polypeptide and a β-catenin polypeptide can be carried out using any of a variety of assays, including, but not limited to, immunological assays (e.g., enzyme-linked immunosorbent assays; radioimmunoassay; and the like); FRET-based assays; BRET-based assays; or any other assay that detects protein-protein binding.

In addition to a NICD polypeptide and a β-catenin polypeptide, and a test agent, a variety of other reagents may be included in the screening assay. These include reagents like salts, neutral proteins, e.g. albumin, detergents, etc., including agents that are used to reduce non-specific or background activity. Reagents that improve the efficiency of the assay, such as protease inhibitors, anti-microbial agents, etc. may be used. The components of the assay mixture are added in any order that provides for the requisite activity. Incubations are performed at any suitable temperature, typically between 4° C. and 40° C. Incubation periods are selected for optimum activity, but may also be optimized to facilitate rapid high-throughput screening. Typically between 0.1 hour and 1 hour will be sufficient.

Assays of the invention include controls, where suitable controls include a sample (e.g., a sample comprising the NICD polypeptide and the β-catenin polypeptide, in the absence of the test agent). Generally a plurality of assay mixtures is run in parallel with different agent concentrations to obtain a differential response to the various concentrations. Typically, one of these concentrations serves as a negative control, i.e. at zero concentration or below the level of detection.

A candidate agent is assessed for any cytotoxic activity (other than anti-proliferative activity) it may exhibit toward a living eukaryotic cell, using well-known assays, such as trypan blue dye exclusion, an MTT (3-(4,5-dimethylthiazol-2-yl)-2,5-diphenyl-2H-tetrazolium bromide) assay, and the like. Agents that do not exhibit cytotoxic activity are considered candidate agents.

In some embodiments, a test agent that inhibits binding between a NICD polypeptide and a β-catenin polypeptide, and that is therefore considered a candidate agent, is further tested for an effect on reducing proliferation of a cancerous cell. Such a test is carried out using well-established methods of measuring cell proliferation. For example, a cancerous cell line is contacted with the candidate agent; and ³H-thymidine incorporation into genomic DNA is measured as an indication of proliferation.

### Detectably Labeled Polypeptides

In some embodiments, one or both of the NICD polypeptide and the β-catenin polypeptide is detectably labeled (“tagged”). Polypeptides modified to comprises a tag and useful in the screening methods of the invention are specifically contemplated herein. By “tag” is meant an attached molecule or molecules useful for the identification or isolation of the attached molecule(s), which can be substrate binding molecules. For example, a tag can be an attachment tag or a label tag. Components having a tag are referred to as “tag-X”, wherein X is the component (e.g., a NICD polypeptide, a β-catenin polypeptide).

The terms “tag”, “detectable label” and “detectable tag” are used interchangeably herein without limitation. In some embodiments, the tag is covalently bound to the attached component. By “tag,” “label,” “detectable label,” or “detectable tag” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Exemplary labels include, but are not limited to, fluorescent labels (e.g. a green fluorescent protein, a red fluorescent protein, a yellow fluorescent protein, etc.) and label enzymes.

Exemplary tags include, but are not limited to, an optically-detectable label, a partner of a binding pair, and a surface substrate binding molecule (or attachment tag). As will be evident to the skilled artisan, many molecules may find use as more than one type of tag, depending upon how the tag is used. In one embodiment, the tag or label as described below is incorporated into the polypeptide as a fusion protein.

As will be appreciated by those in the art, tag-components of the invention can be made in various ways, depending largely upon the form of the tag. Components of the invention and tags are preferably attached by a covalent bond. Examples of tags are described below.

### Detectably Labeled Polypeptides

In some embodiments, one or both of the NICD polypeptide and the β-catenin polypeptide is detectably labeled (“tagged”). Polypeptides modified to comprises a tag and useful in the screening methods of the invention are specifically contemplated herein. By “tag” is meant an attached molecule or molecules useful for the identification or isolation of the attached molecule(s), which can be substrate binding molecules. For example, a tag can be an attachment tag or a label tag. Components having a tag are referred to as “tag-X”, wherein X is the component (e.g., a NICD polypeptide, a β-catenin polypeptide).

The terms “tag”, “detectable label” and “detectable tag” are used interchangeably herein without limitation. In some embodiments, the tag is covalently bound to the attached component. By “tag,” “label,” “detectable label,” or “detectable tag” is meant a molecule that can be directly (i.e., a primary label) or indirectly (i.e., a secondary label) detected; for example a label can be visualized and/or measured or otherwise identified so that its presence or absence can be known. As will be appreciated by those in the art, the manner in which this is performed will depend on the label. Exemplary labels include, but are not limited to, fluorescent labels (e.g. a green fluorescent protein, a red fluorescent protein, a yellow fluorescent protein, etc.) and label enzymes.

Exemplary tags include, but are not limited to, an optically-detectable label, a partner of a binding pair, and a surface substrate binding molecule (or attachment tag). As will be evident to the skilled artisan, many molecules may find use as more than one type of tag, depending upon how the tag is used. In one embodiment, the tag or label as described below is incorporated into the polypeptide as a fusion protein.

As will be appreciated by those in the art, tag-components of a subject screening assay can be made in various ways, depending largely upon the form of the tag. Components of the invention and tags can be attached by a covalent bond. Examples of tags are described below.

### Exemplary Tags Useful in the Invention

In one embodiment, the tag is a polypeptide which is provided as a portion of a chimeric molecule comprising a first polypeptide fused to another, heterologous polypeptide or amino acid sequence. In one embodiment, such a chimeric molecule comprises a fusion of a first polypeptide with a tag polypeptide. The tag is generally placed at the amino- or carboxyl-terminus of the polypeptide. In embodiments in which the tagged polypeptide is to be used in a cell-based assay and is to be expressed a recombinant protein, the tag is usually a genetically encodable tag (e.g., fluorescent polypeptide, immunodetectable polypeptide, and the like).

The tag polypeptide can be, for example, an immunodetectable label (i.e., a polypeptide or other moiety which provides an epitope to which an anti-tag antibody can selectively bind), a polypeptide which serves as a ligand for binding to a receptor (e.g., to facilitate immobilization of the chimeric molecule on a substrate); an enzyme label (e.g., as described further below); or a fluorescent label (e.g., as described further below). Tag polypeptides provide for, for example, detection using an antibody against the tag polypeptide, and/or a ready means of isolating or purifying the tagged polypeptide (e.g., by affinity purification using an anti-tag antibody or another type of receptor-ligand matrix that binds to the tag). The production of tag-polypeptides by recombinant means is within the knowledge and skill in the art.

Production of immunodetectably-labeled proteins (e.g., use of FLAG, HIS (e.g., poly(histidine), such as His₆), and the like, as a tag) is well known in the art and kits for such production are commercially available (for example, from Kodak and Sigma). See, e.g., Winston et al., Genes and Devel. 13:270-283 (1999), incorporated herein in its entirety, as well as product handbooks provided with the above-mentioned kits. Production of proteins having His-tags by recombinant means is well known, and kits for producing such proteins are commercially available. Such a kit and its use is described in the QIAexpress Handbook from Qiagen by Joanne Crowe et al., hereby expressly incorporated by reference.

Methods for production of polypeptides having an optically-detectable label are well known. An “optically detectable label” includes labels that are detectably due to inherent properties (e.g., a fluorescent label), or which may be reacted with a substrate or act as a substrate to provide an optically detectable (e.g., colored) reaction product (e.g., horse radish peroxidase).

By “fluorescent label” is meant any molecule that may be detected via its inherent fluorescent properties, which include fluorescence detectable upon excitation. Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 2002 Molecular Probes Handbook, 9th Ed., by Richard P. Haugland, hereby expressly incorporated by reference.

Suitable fluorescent labels include, but are not limited to, green fluorescent protein (GFP; Chalfie, et al., Science 263(5148):802-805 (Feb. 11, 1994); and enhanced GFP (EGFP); Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), β-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558), a GFP from species such as Renilla reniformis, Renilla mulleri, or Ptilosarcus guernyi, as described in, e.g., WO 99/49019 and Peelle et al. (2001) J. Protein Chem. 20:507-519; “humanized” recombinant GFP (hrGFP) (Stratagene); any of a variety of fluorescent and colored proteins from Anthozoan species, as described in, e.g., Matz et al. (1999) Nature Biotechnol. 17:969-973; U.S. Patent Publication No. 2002/0197676, or U.S. Patent Publication No. 2005/0032085; and the like.

In some instances, multiple fluorescent labels are employed. In one embodiment, at least two fluorescent labels are used which are members of a fluorescence resonance energy transfer (FRET) pair. FRET can be used to detect association/dissociation of for example, a NICD polypeptide and a β-catenin polypeptide; and the like. In general, such FRET pairs are used in in vitro assays.

FRET is phenomenon known in the art wherein excitation of one fluorescent dye is transferred to another without emission of a photon. A FRET pair consists of a donor fluorophore and an acceptor fluorophore (where the acceptor fluorophore may be a quencher molecule). The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Forster radius, which is typically 10-100 angstroms. Changes in the fluorescence emission spectrum comprising FRET pairs can be detected, indicating changes in the number of that are in close proximity (i.e., within 100 angstroms of each other). This will typically result from the binding or dissociation of two molecules, one of which is labeled with a FRET donor and the other of which is labeled with a FRET acceptor, wherein such binding brings the FRET pair in close proximity.

Binding of such molecules will result in an increased fluorescence emission of the acceptor and/or quenching of the fluorescence 15 emission of the donor. FRET pairs (donor/acceptor) useful in the invention include, but are not limited to, EDANS/fluorescein, IAEDANS/fluorescein, fluorescein/tetramethylrhodamine, fluorescein/Cy 5, IEDANS/DABCYL, fluorescein/QSY-7, fluorescein/LC Red 640, fluorescein/Cy 5.5 and fluorescein/LC Red 705.

In another aspect of FRET, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

The skilled artisan will appreciate that FRET and fluorescence quenching allow for monitoring of binding of labeled molecules over time, providing continuous information regarding the time course of binding reactions. It is important to remember that attachment of labels or other tags should not interfere with active groups on the interacting polypeptides. Amino acids or other moieties may be added to the sequence of a protein, through means well known in the art and described herein, for the express purpose of providing a linker and/or point of attachment for a label. In one embodiment, one or more amino acids are added to the sequence of a component for attaching a tag thereto, with a fluorescent label being of particular interest.

In other embodiments, detection involves bioluminescence resonance energy transfer (BRET). BRET is a protein-protein interaction assay based on energy transfer from a bioluminescent donor to a fluorescent acceptor protein. The BRET signal is measured by the amount of light emitted by the acceptor to the amount of light emitted by the donor. The ratio of these two values increases as the two proteins are brought into proximity. The BRET assay has been amply described in the literature. See, e.g., U.S. Pat. Nos. 6,020,192; 5,968,750; and 5,874,304; and Xu et al. (1999) Proc. Natl. Acad. Sci. USA 96:151-156. BRET assays may be performed by analyzing transfer between a bioluminescent donor protein and a fluorescent acceptor protein. Interaction between the donor and acceptor proteins can be monitored by a change in the ratio of light emitted by the bioluminescent and fluorescent proteins.

Alternatively, binding may be assayed by fluorescence anisotropy. Fluorescence anisotropy assays are amply described in the literature. See, e.g., Jameson and Sawyer (1995) Methods Enzymol. 246:283-300.

By “label enzyme” is meant an enzyme which may be reacted in the presence of a label enzyme substrate which produces a detectable product. Suitable label enzymes also include optically detectable labels (e.g., in the case of HRP). Suitable label enzymes for use in the present invention include but are not limited to, horseradish peroxidase (HRP), alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By “radioisotope” is meant any radioactive molecule. Suitable radioisotopes for use in the invention include, but are not limited to ¹⁴C, ³H, ³²P, ³³P, ³⁵S, ¹²⁵I, and ¹³¹I. The use of radioisotopes as labels is well known in the art.

In addition, labels may be indirectly detected, that is, the tag is a partner of a binding pair. By “partner of a binding pair” is meant one of a first and a second moiety, wherein said first and said second moiety have a specific binding affinity for each other. Suitable binding pairs for use in the invention include, but are not limited to, antigen/antibodies (for example, digoxigenin/anti-digoxigenin, dinitrophenyl (DNP)/anti-DNP, dansyl-X-anti-dansyl, fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide (Hopp et al., BioTechnol, 6:1204-1210 (1988)); the KT3 epitope peptide (Martin et al., Science, 255:192-194 (1992)); tubulin epitope peptide (Skinner et al., J. Biol. Chem., 266: 15 163-15 166 (1991)); and the T7 gene 10 protein peptide tag (Lutz-Freyemuth et al., Proc. Natl. Acad. Sci. USA, a:6393-6397 (1990)) and the antibodies each thereto. Generally, in one embodiment, the smaller of the binding pair partners serves as the tag, as steric considerations in ubiquitin ligation may be important. As will be appreciated by those in the art, binding pair partners may be used in applications other than for labeling, such as immobilization of the protein on a substrate and other uses as described below.

As will be appreciated by those in the art, a partner of one binding pair may also be a partner of another binding pair. For example, an antigen (first moiety) may bind to a first antibody (second moiety) which may, in turn, be an antigen for a second antibody (third moiety). It will be further appreciated that such a circumstance allows indirect binding of a first moiety and a third moiety via an intermediary second moiety that is a binding pair partner to each. As will be appreciated by those in the art, a partner of a binding pair may comprise a label, as described above. It will further be appreciated that this allows for a tag to be indirectly labeled upon the binding of a binding partner comprising a label. Attaching a label to a tag which is a partner of a binding pair, as just described, is referred to herein as “indirect labeling.”

In one embodiment, the tag is surface substrate binding molecule. By “surface substrate binding molecule” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the influenza hemagglutinin (HA) tag polypeptide and its antibody 12CA5 substrate (Field et al., Mol. Cell. Biol., 8:2159-2165 (1988)); the c-myc tag and the 8F9, 3C7, 6E107 G4, B7 and 9E10 antibody substrates thereto (Evan et al., Molecular and Cellular Biol, 5:3610-3616 (1985)]; and the Herpes Simplex virus glycoprotein D (gD) tag and its antibody substrate (Paborsky et al., Protein Engineering, 3(6):547-553 (1990)). In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj, Chem. 2:342-348 (1991); Torchilin et al., supra; Trubetskoy et al., Bioconi. Chem. 33323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available and production of proteins with CBP is described in Simcox et al., Strategies 8:40-43 (1995), which is hereby incorporated by reference in its entirety.

Where appropriate, functionalization of labels with chemically reactive groups such as thiols, amines, carboxyls, etc. is generally known in the art. In one embodiment, the tag is functionalized to facilitate covalent attachment.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see, e.g., chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known. Methods for labeling of proteins with radioisotopes are known in the art. For example, such methods are found in Ohta et al., Molec. Cell 3:535-541 (1999), which is hereby incorporated by reference in its entirety.

The covalent attachment of the tag may be either direct or via a linker. In one embodiment, the linker is a relatively short coupling moiety that is used to attach the molecules. A coupling moiety may be synthesized directly onto a component of the invention, ubiquitin for example, and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer.

In one embodiment, the covalent attachment is direct, that is, no linker is used. In this embodiment, the component can contain a functional group such as a carboxylic acid which is used for direct attachment to the functionalized tag. It should be understood that the component and tag may be attached in a variety of ways, including those listed above. What is important is that manner of attachment does not significantly alter the functionality of the component. For example, in tag-NICD, the tag should be attached in such a manner as to allow binding between a NICD polypeptide and a β-catenin polypeptide.

As will be appreciated by those in the art, the above description of covalent attachment of a label and NICD applies equally to the attachment a label to a β-catenin polypeptide. In one embodiment, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of a NICD or a β-catenin protein, amino modified or hydrazine modified tags will be used for coupling via carbodimide chemistry, for example using 1-ethyl-3-(3-dimethylaminopropyl)-carbodimide (EDAC) as is known in the art. In one embodiment, the carbodiimide is first attached to the tag, such as is commercially available for many of the tags described herein.

Methods of Identifying an Agent that Inhibits Cleavage of the Intracellular Domain of a Notch1 Polypeptide from the Transmembrane Domain of the Notch1 Polypeptide

The present disclosure provides an in vitro method of identifying an agent that reduces cleavage of the intracellular domain of a Notch1 polypeptide from the transmembrane domain of the Notch1 polypeptide. The method generally involves: a) contacting a Notch1 polypeptide that comprises the transmembrane domain and the intracellular domain of a Notch1 polypeptide with a test agent and an enzyme that cleaves the intracellular domain of the Notch1 polypeptide from the transmembrane domain of the Notch1 polypeptide; and b) determining the effect, if any, of the test agent on cleavage of the intracellular domain of the Notch1 polypeptide from the transmembrane domain of the Notch1 polypeptide mediated by the enzyme. An agent that reduces the cleavage by at least about 10% is considered a candidate agent for reducing cell proliferation.

An enzyme that cleaves the intracellular domain of the Notch1 polypeptide from the transmembrane domain of the Notch1 polypeptide is generally a γ-secretase. Gamma-secretases are known in the art.

In some embodiments, the assay is carried out in a cell-free assay system. In other embodiments, the assay is carried out in a living cell, e.g., a eukaryotic cell such as a mammalian cell or a mammalian cell line.

A Notch1 polypeptide suitable for use in a subject screening method can comprise an amino acid sequence having at least about 85%, at least about 90%, at least about 95%, at least about 98%, at least about 99%, or 100%, amino acid sequence identity to a contiguous stretch of at least 25 amino acids, at least 50 aa, at least 100 aa, at least 200 aa, at least 300 aa, at least 400 aa, at least 500 aa, at least 600 aa, at least 700 aa, at least about 800 aa, of amino acids 1737-2556 of the amino acid sequence set forth in SEQ ID NO:1 and depicted in FIGS. 7A and 7B. In some embodiments, the Notch1 polypeptide lacks extracellular domains.

Whether a test agent inhibits cleavage of the intracellular domain of the Notch1 polypeptide from the transmembrane domain of the Notch1 polypeptide can be determined by detecting the cleaved intracellular domain of the Notch1 polypeptide.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

### Notch Post-Translationally Regulates β-Catenin Protein in Stem Cells and Human Cancer Cells

### Experimental Procedures

### Mouse Genetics and Cell Culture

The Isl1^{Cre}; RBP-J^{flox/flox} or Isl1^{Cre}; Notch1^{flox/flox} embryos were obtained by crossing Isl^{Cre}; RBP-J^{flox/+} mice with RBP-J^{flox/flox} or Notch1^{tm2Rko} mice, respectively (Srinivas et al., 2001; Yang et al., 2004). Isl1^{Cre}; β-Catenin(ex3)^{loxP/+} or Isl1^{Cre}; β-Catenin(ex3)^{loxP/+} Gt(ROSA)26Sor^{tm1(Notch1)Dam}/J embryos were obtained by crossing Isl^{Cre} mice with β-Catenin (ex3)^{loxP/+}, Gt(ROSA)26Sor^{tm1(Notch1)Dam}/J mice (Murtaugh et al., 2003).

Constructs, siRNA, Transfection, Gene Expression and Luciferase Assays

For Notch, RBP-J or Numb/Numbl knockdown experiments, Notch1-4, RBPSUH or Numb/Numbl On-TARGETplus SMARTpool (Dharmacon L-041110, L-044202, L-047867, L-046498, L-007772 or L-046935/L-046983) or Block-iT Alexa Fluor Red (46-5, 318, Invitrogen) was used at concentrations of 50, 100, or 200 nM for cell transfection. Tethered and truncated forms of Notch1 constructs were kindly provided by Drs. R. Kopan (Washington University, St. Louis, Mo.) and M. Nakafuku (Cincinnati Children's Hospital, Cincinnati, Ohio), respectively. Cells were transfected with Lipofectamine LTX (Invitrogen) or Lipofectamine 2000 (Invitrogen) in single-cell suspensions. For gene expression analysis, qPCR was performed with the ABI Prism system (7900HT, Applied Biosystems) with the following primers: β-Catenin (Mm01350394_m1), Cyclin D1 (Mm00432359_m1), Gapdh (Mm99999915_g1). All samples were run at least in triplicate. Real-time quantitative PCR data were normalized and standardized with SDS2.2 software. The constructs to measure Notch/RBP-J (JH23A) were kindly provided by Dr. N. Gaiano (Johns Hopkins University). For luciferase assays, Renilla was used as an internal normalization control.

### Co-Immunoprecipitation and Western Analyses

Cells were transfected with indicated constructs and cultured for 24 hours (with/without BIO, 2 μM). Cells were scraped off the 100-mm dish and lysed in 1 ml of lysis buffer (1 mM PMSF, 1 mM EDTA, 10 mM Tris-HCl, 0.1% Triton X100, 1× Complete Protease Inhibitor Cocktail (Roche) in PBS). The lysates were spun down, and 1 μg of anti-c-Myc antibody (Sigma, M4439) or anti-Flag antibody (Sigma, F1804) was added to 500 μl of the supernatant. A 50-50 mixture of protein A Sepharose (Amersham) and protein G Sepharose (Amersham) was added to the lysate/antibody mixture for immunoprecipitation for 1 hour. The resulting outputs were washed with lysis buffer and subjected to western blot analysis. For western blotting, samples were analyzed using antibodies against active-β-Catenin (anti-ABC, Millipore), phospho-β-Catenin (Ser33/37/Thr41, Cell Signaling), β-Catenin (sc-1496, Santa Cruz Biotechnology) and Gapdh (Santa Cruz Biotechnology).

### DAPT, Ibuprofen and BIO Treatment

Cells were treated with DAPT (CALBIOCHEM Cat#565784), ibuprofen (99% pure, Sigma) or BIO (CALBIOCHEM Cat#361550) at the indicated concentrations.

### Statistical Analyses

The two-tailed Student's t-test, type II, was used for data analyses. P<0.05 was considered significant.

### Results

### Notch Negatively Regulates Active β-Catenin Protein Levels in Multiple Stem Cells

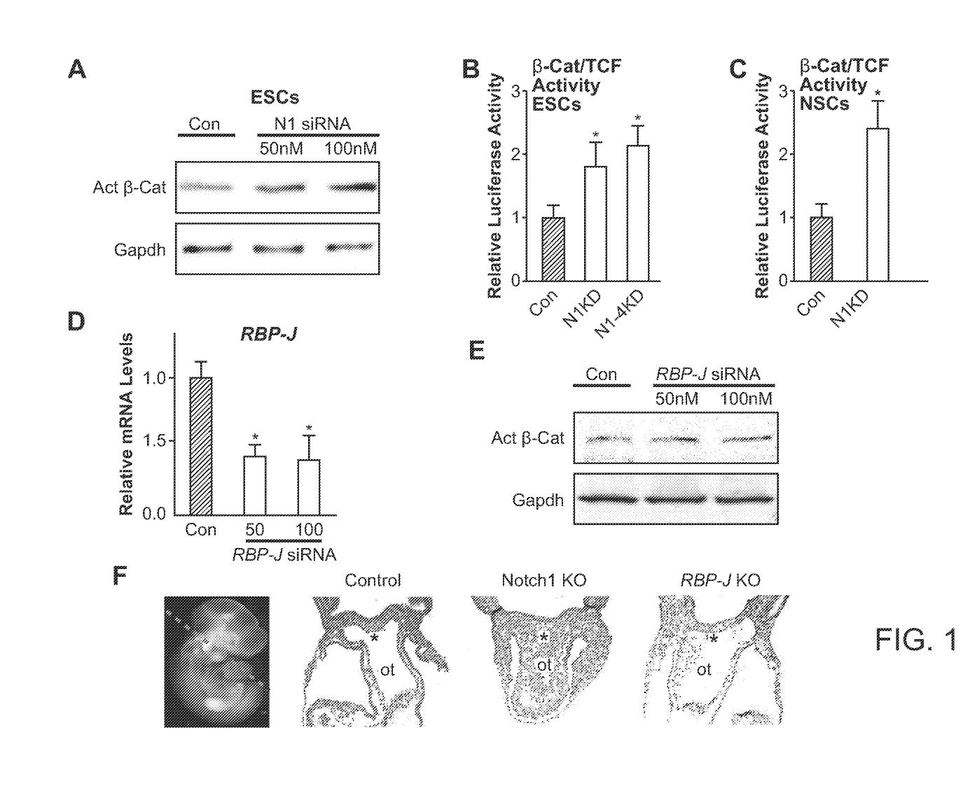

To determine if Notch negatively regulates β-Catenin protein levels in embryonic stem cells (ESCs), a Notch1 siRNA was used to decrease Notch1 levels. It was found that reduced Notch1 levels resulted in an increase in protein levels of the dephosphorylated, transcriptionally active form of β-Catenin (FIG. 1A). In agreement with this finding, the Notch1-knockdown (KD) ESCs showed significantly more TCF/β-Catenin-dependent luciferase activity than controls (FIG. 1B). Moreover, knocking down transcripts of all four Notch receptors (Notch1, 2, 3, 4) by applying Notch1-4 siRNAs further increased β-Catenin activity (FIG. 1B). The increase was also observed in Notch siRNA-treated neural stem cells (NSCs) (FIG. 1C) and in mouse cardiac progenitor cells (CPCs) lacking Notch1 in vivo and in vitro (Kwon et al., 2009), suggesting that Notch functions broadly to negatively regulate active β-Catenin protein in stem cell populations.

It was determined if the regulation of β-Catenin protein occurs through the canonical Notch signaling pathway involving the transcription factor, RBP-J. An RBP-J-specific siRNA was introduced into ESCs to reduce RBP-J levels. Despite a ˜70% KD of RBP-J mRNA (FIG. 1D), active β-Catenin levels were unchanged (FIG. 1E). To determine if RBP-J mediates the Notch regulation of β-Catenin in vivo, Notch1 or RBP-J was deleted in CPCs by inter-crossing Notch1^{tm2Rko} (Yang et al., 2004) or RBP-J^{flox/flox} mice (Tanigaki et al., 2002) with mice containing Cre recombinase in the Isl1 locus (Isl1^{Cre}) (Srinivas et al., 2001). Isl1 marks an undifferentiated pool of CPCs (Bu et al., 2009; Cai et al., 2003), whose expansion depends on Wnt/β-Catenin signaling (Kwon et al., 2007; Qyang et al., 2007). Unlike embryos with a Notch1 deletion, the resulting RBP-J mutant embryos showed no expansion of CPCs (FIG. 1F). These data suggest that Notch-mediated regulation of active β-Catenin protein in ESCs and CPCs does not involve RBP-J-dependent transcriptional regulation.

### Notch Regulates β-Catenin Protein Levels Independent of the β-Catenin Destruction Complex

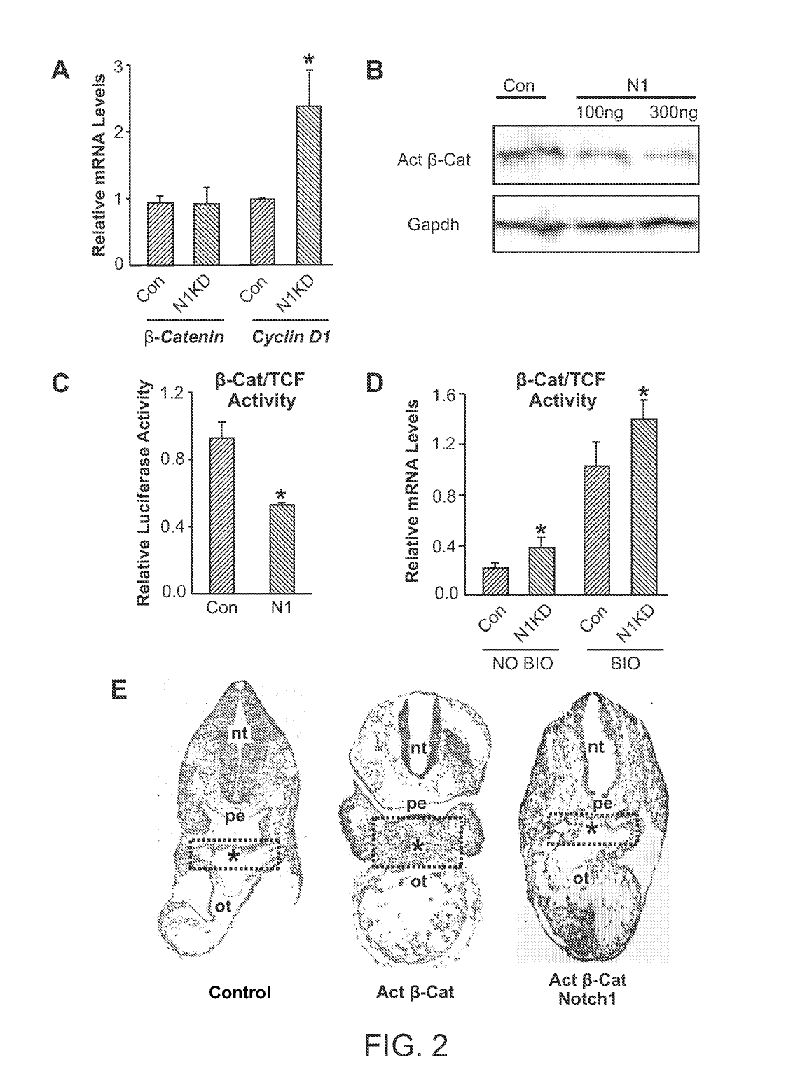

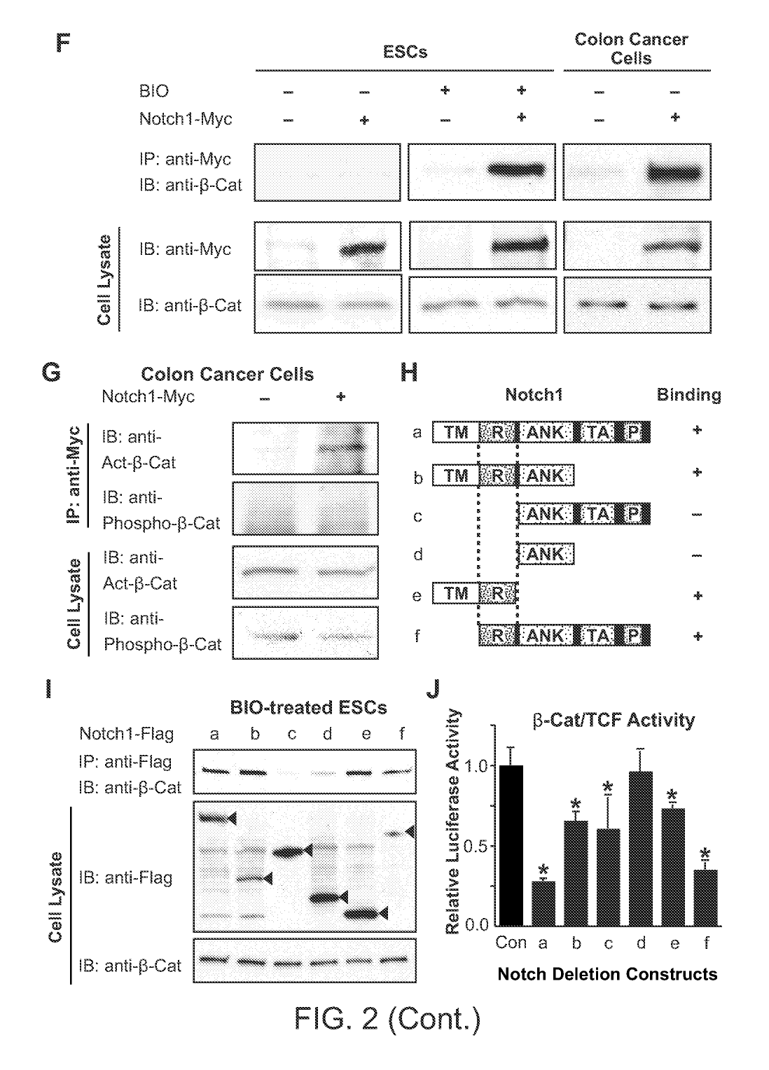

RBP-J-independent Notch signaling has been described in vertebrates and invertebrates (Martinez Arias et al., 2002) and is thought to involve Notch-mediated transcription through other DNA-binding proteins. However, quantitative polymerase chain reaction (qPCR) revealed that levels of β-Catenin transcripts were not altered in Notch1 KD ESCs, although Cyclin D1, a direct target of TCF/β-Catenin (Tetsu and McCormick, 1999), was significantly upregulated in Notch1 KD cells (FIG. 2A). This raised the possibility that Notch affects β-Catenin protein at the post-translational level through the β-Catenin destruction complex.

To examine whether Notch utilizes with the destruction complex to negatively regulate active β-Catenin protein, we used a pharmacological glycogen synthase kinase-3β (GSK3β) inhibitor, 6-bromoindirubin-3′-oxime (BIO). BIO specifically inhibits GSK3β activity and inactivates the destruction complex, resulting in the accumulation of active β-Catenin (Meijer et al., 2003). Overexpression of the Notch1 intracellular domain (N1ICD) in ESCs decreased active β-Catenin protein levels and activity even in the presence of BIO (FIGS. 2B and 2C). Furthermore, reduced levels of Notch1 increased β-Catenin activity even beyond that seen in BIO-treated ESCs (FIG. 2D). This suggests that Notch regulation of β-Catenin protein in vitro may be independent of the destruction complex involving adenomatous polyposis coli (APC) and GSK3β.

To determine if Notch suppresses β-Catenin activity independent of the destruction complex in vivo, a form of β-Catenin that cannot be degraded by the destruction complex was expressed, with or without Notch1, in mouse CPCs. This was done by crossing Isl1^{Cre} mice with mice containing loxP sites surrounding exon 3 of β-catenin (β-Catenin(ex3)^{loxP}) (Harada et al., 1999), required for APC-mediated degradation, with or without mice overexpressing Notch1 (Gt(ROSA)26Sor^{tm1(Notch1)Dam}/J) (Murtaugh et al., 2003). Unlike the previously reported expansion of precardiac mesoderm induced by expression of stabilized β-Catenin in CPCs (Kwon et al., 2007), co-expression of stabilized β-Catenin and Notch1 completely abolished the β-Catenin-mediated expansion of precardiac mesoderm (FIG. 2E). These findings implied that Notch does not require the destruction complex to negatively regulate β-Catenin protein levels in CPCs in vivo.

Notch Physically Associates with Active β-Catenin through the RAM Domain

Given that Notch does not require the β-Catenin destruction complex to regulate β-Catenin protein, it was examined if Notch modulates active β-Catenin protein levels through a direct physical interaction. To do this, Myc-tagged N1ICD was expressed in ESCs; and co-immunoprecipitation (Co-IP) assays were performed with anti-Myc antibodies with or without BIO. No detectable interaction of endogenous β-Catenin with Notch1 was observed in the absence of BIO (FIG. 2F). However, when treated with BIO, which greatly increases active β-Catenin levels by inactivating the destruction complex, Notch1 co-precipitated with endogenous β-Catenin (FIG. 2F), but not with APC, Axin, Gsk3β or TrCP. This suggested that Notch selectively interacts with active, unphosphorylated β-Catenin, whose levels are normally very low in ESCs. To investigate this possibility further, a human colon cancer cell line, SW480, was used. SW480 contains high levels of active β-Catenin due to an APC mutation that causes colon cancer (Korinek et al., 1997). When expressed in SW480 cells, Notch strongly associated with endogenous β-Catenin even without BIO treatment (FIG. 2F). Further analysis of the precipitated β-Catenin confirmed enrichment of active, unphosphorylated, β-Catenin, but not of N-terminal phosphorylated β-Catenin (FIG. 2G). These data suggest that Notch physically associates with the active, unphosphorylated, β-Catenin.

Next, the domains of Notch responsible for β-Catenin association were mapped by performing Co-IP experiments with a series of truncated Notch mutants that lacked the extracellular domain (Yamamoto et al., 2001) (FIG. 2H). It was found that Notch mutants lacking the RAM domain could not associate with β-Catenin (FIG. 2I). To determine if the RAM domain, also required for RBP-J interaction (Tamura et al., 1995), was necessary for Notch regulation of active β-Catenin activity, control and mutant Notch constructs were expressed in BIO-treated ESCs. Increased expression of Notch with the RAM domain significantly decreased β-Catenin activity, although other domains may also contribute to repression (FIG. 2J). Thus, the RAM domain was necessary for β-Catenin interaction and for full suppression of β-Catenin activity.

### Membrane Cleavage of Notch is not Necessary to Regulate Active β-Catenin Protein Levels

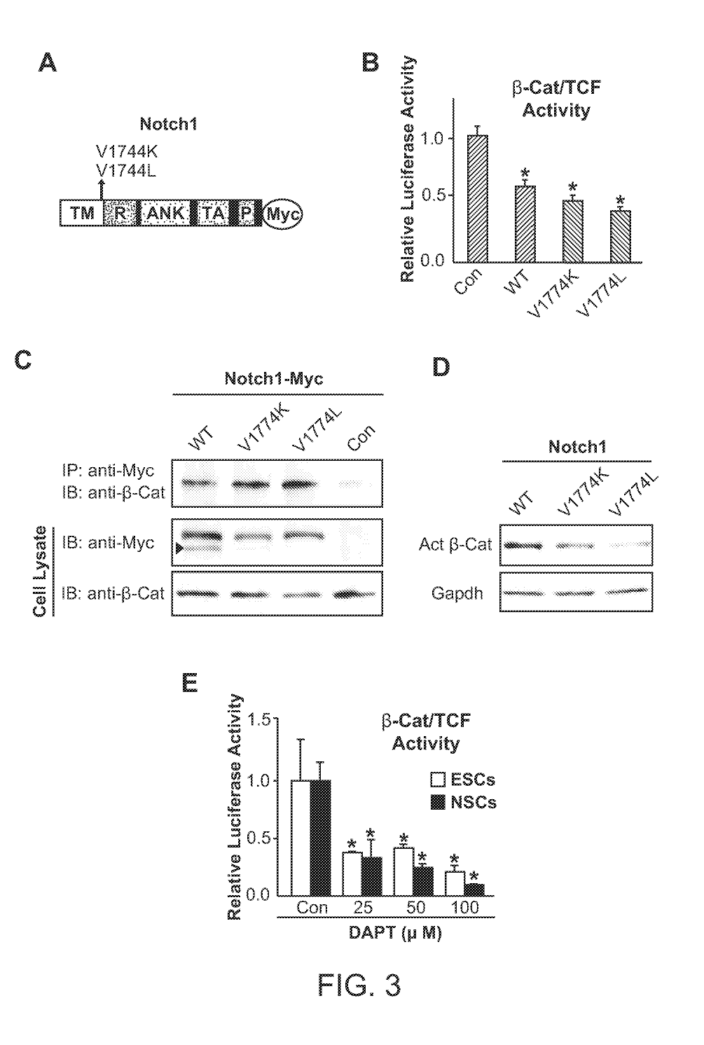

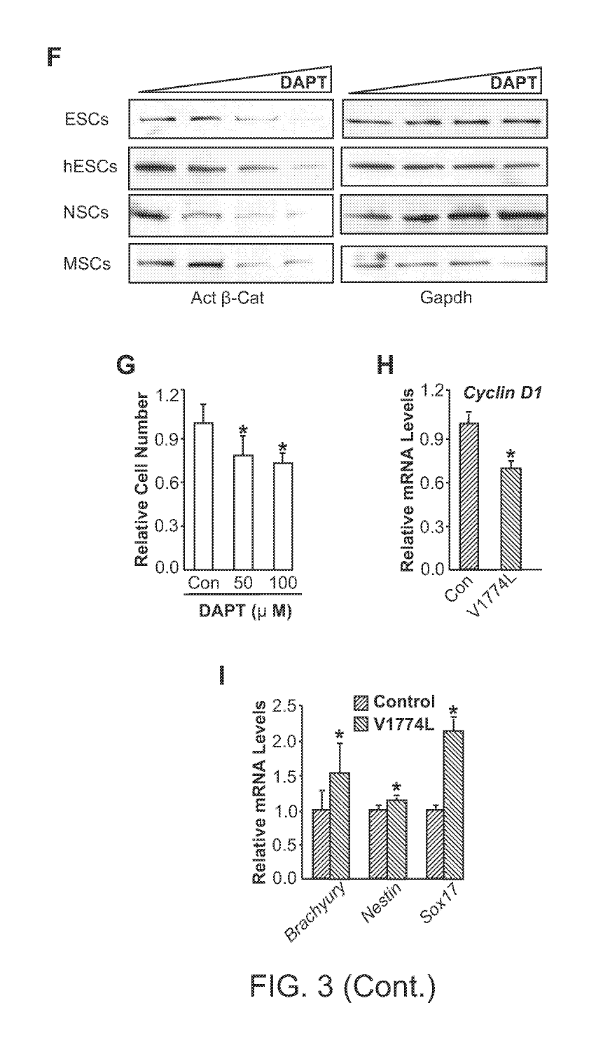

It was further investigated whether ligand-dependent cleavage of Notch to free the NICD, which is essential for canonical Notch signaling, was necessary for the Notch regulation of active β-Catenin protein. Notch1 intracellular cleavage occurs between amino acids G1743 and V1744 in a highly conserved manner; mutations of V1744 (V1744K or V1744L) block intracellular cleavage, leaving Notch tethered to the membrane (Schroeter et al., 1998) (FIG. 3A). Constitutively activated membrane-bound Notch1 was expressed in ESCs with or without mutations at V1744 (FIG. 3B). Expression of the tethered forms of Notch decreased β-Catenin transcriptional activity similar to, and slightly more than, wildtype Notch (FIG. 3B). In addition, more endogenous active β-Catenin was immunoprecipitated with the tethered forms of Notch than with the wild-type Notch (FIG. 3C). In agreement with the Co-IP result, active β-Catenin protein levels were considerably lower in cells with tethered forms of Notch than those with control Notch (FIG. 3D).

To determine if endogenous membrane-bound Notch negatively regulates active β-Catenin protein, Notch endoproteolysis, which is mediated by the presenilin-γ-secretase complex that intracellularly cleaves membrane-bound Notch (De Strooper et al., 1999), was blocked. It was found that ESCs treated with the γ-secretase inhibitor (GSI), DAPT (N—[N-(3,5-difluorophenacetyl)-L-alanyl]-S-phenylglycine t-butyl ester) (Sastre et al., 2001), had a significant reduction of active β-Catenin activity and protein levels in a dose-dependent fashion (FIGS. 3E and 3F), resulting in a significant decrease in cell numbers (FIG. 3G). This trend was also observed in hESCs, NSCs and bone marrow mesenchymal stem cells (FIGS. 3E and 3F). It was concluded that endogenous membrane-bound Notch physically interacts with and negatively regulates active β-Catenin protein accumulation in multiple stem cell populations.

### Increased Levels of Membrane-Bound Notch Favor ESC Differentiation

Wnt/β-Catenin signaling has long been implicated in maintenance and self-renewal of stem cells. This observation was used to test whether membrane-bound Notch had biological activity and could affect Wnt/β-Catenin-dependent ESC maintenance. It was found that cells expressing tethered Notch (V1744L) had decreased levels of a β-Catenin target gene, Cyclin D1, whose expression was elevated in Notch1-deficient ESCs (FIGS. 2A and 3H). Importantly, expression of endodermal and mesodermal genes (Sox17 and Brachyury, respectively) was upregulated in cells with tethered Notch, even in the presence of leukemia inhibitory factor (LIF) (FIG. 31). The magnitude of the effect correlated inversely with concentrations of leukemia inhibitory factor (LIF), a factor that promotes ESC pluripotency. These findings suggest that increased levels of membranous Notch could push ESCs toward a more differentiated state.

### Membrane-Bound Notch-Mediated Degradation of β-Catenin in ESCs Requires Numb

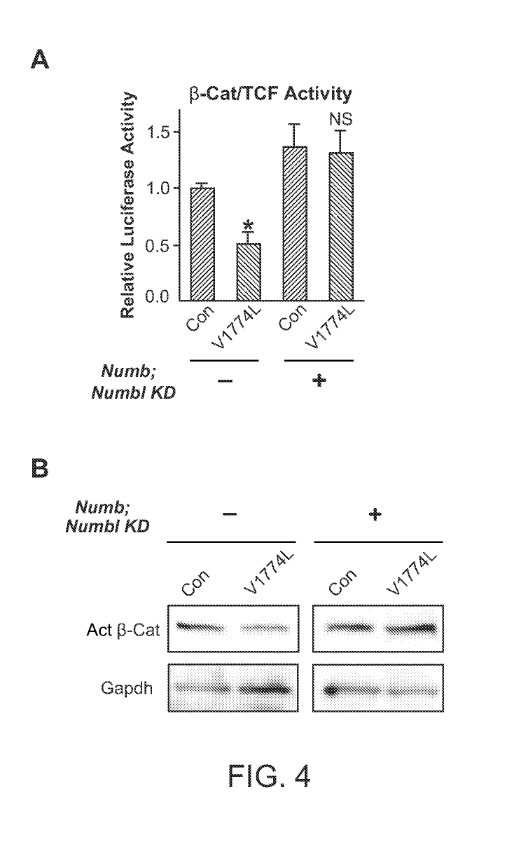

Membrane-bound Notch is regulated by endosomal sorting pathways, leading to recycling or lysosomal degradation (Kanwar and Fortini, 2004; Lu and Bilder, 2005). In Drosophila, the conserved endocytic adaptor protein Numb, which is present as two homologues Numb and Numbl in mammals, negatively regulates Notch protein (Guo et al., 1996; Zhong et al., 1996; Zhong et al., 1997). One mechanism by which Numb inhibits Notch signaling is by trafficking membrane-bound Notch into the lysosome for degradation (McGill et al., 2009). To determine if Numb activity is required for degradation of active β-Catenin complexed with membrane-bound Notch, Numb and Numbl levels were knocked down in ESCs in the presence of the tethered form of Notch (V1744L). Tethered Notch failed to suppress β-Catenin activity in Numb and Numbl-deficient ESCs (FIG. 4A). Consistent with this, active β-Catenin protein levels were no longer affected by tethered Notch upon knockdown of Numb and Numbl (FIG. 4B). These data suggest that Numb and Numbl may be involved in trafficking the Notch-β-Catenin complex for degradation, which may occur in the lysosome.

### DAPT Treatment Decreases Active β-Catenin Levels in Human Colon Cancer Cells

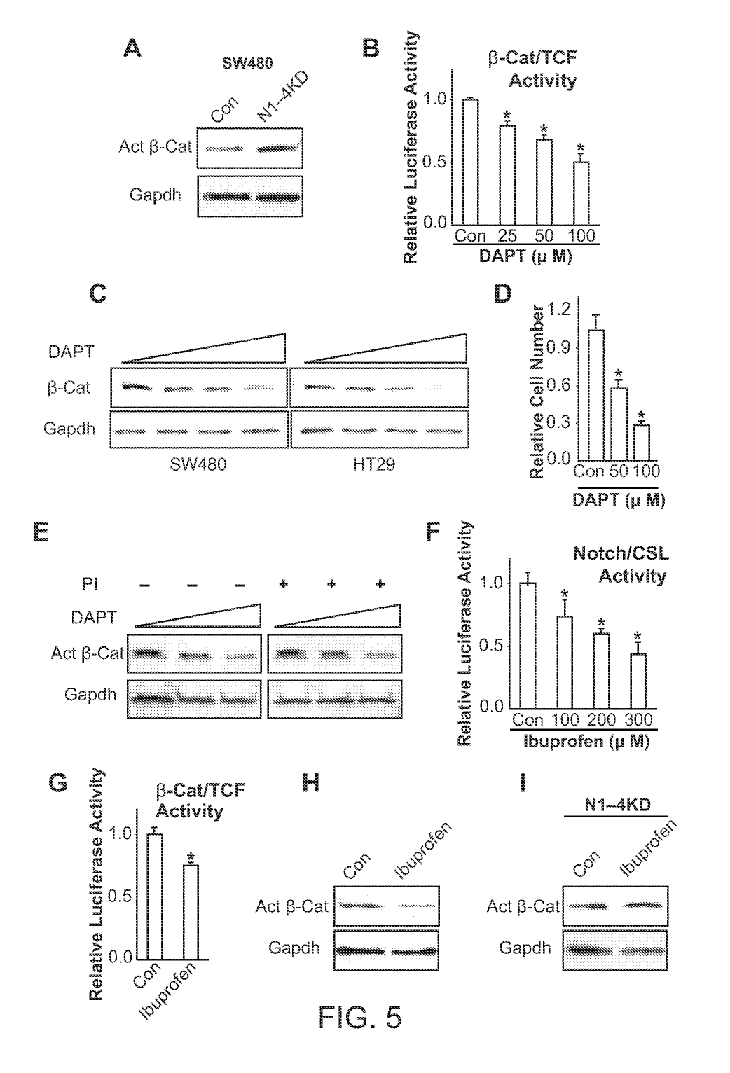

Upregulation of active β-Catenin levels is an important oncogenic step in a number of cancers (Peifer and Polakis, 2000). Extrapolating the results from stem/progenitor cells, it was hypothesized that membrane-bound Notch could affect β-Catenin levels in APC-mutated human cancer cells containing markedly elevated active β-Catenin protein. Notch 1-4 was knocked down in SW480 human colorectal cancer cells and found a prominent increase in active β-Catenin protein levels (FIG. 5A). This result provided additional evidence for regulation of β-Catenin by Notch independent of the destruction complex. Conversely, treatment of two human colorectal cancer cell lines, SW480 and HT-29, with DAPT, which prevents NICD cleavage, resulted in a dose-dependent decrease in β-Catenin protein, TCF/β-Catenin-dependent transcriptional activity, and cell expansion (FIGS. 5B-D). Proteasome inhibitors that block the destruction complex-mediated degradation of β-Catenin resulted in increased active β-Catenin levels as expected but failed to prevent the Notch-mediated decrease in β-Catenin protein (FIG. 5E). This indicates that Notch regulation of β-Catenin protein is unlikely proteasome-mediated and supports the earlier evidence showing Numb-dependence and potential involvement of the lysosome.

Ibuprofen Lowers β-Catenin Levels through Notch in Human Colon Cancer Cells

A subset of non-steroidal-anti-inflammatory drugs (NSAIDs) also has significant GSI activity (Eriksen et al., 2003), and chronic use of NSAIDs in humans has frequently been reported to lower the risk of developing primary and recurrent colorectal cancer (Chan et al., 2005; Rostom et al., 2007). Although the anti-neoplastic effects of NSAIDs were attributed to their anti-inflammatory function of inhibiting Cycloxygenase 2 (COX-2), NSAIDs surprisingly also slow proliferation of COX-2-deficient colorectal cancer cells such as SW480 cells (Bottone et al., 2003; Shiff et al., 1995). It was found that Ibuprofen treatment resulted in a dose-dependent decrease of canonical Notch transcriptional activity, determined by Notch/RBP-J-dependent luciferase activity, confirming its γ-secretase inhibitor (GSI) activity (FIG. 5F). Ibuprofen treatment also lowered levels of active β-Catenin transcriptional activity and protein (FIGS. 5G and 5H). Importantly, the reduction of β-Catenin protein levels upon Ibuprofen treatment of cancer cells was not observed after knockdown of Notch1-4 (FIG. 4I). This suggests that NSAIDs act, at least in part, through Notch to decrease active β-Catenin protein levels, and this regulation may contribute to the overall protective effects of NSAIDs on colorectal cancers. This result is consistent with the observation that GSI treatment in APC mutant mice reduces proliferating adenomas in the intestine (Koch and Radtke, 2007; van Es et al., 2005).

FIGS. 1A-F. Notch Negatively Regulates Active β-Catenin in Stem Cells Independently of RBP-J (A) Western analysis of ESCs transfected with control or Notch1 (N1) siRNA (50 or 100 nM) with active β-Catenin (Act β-Cat) antibody that detects N-terminal-dephosphorylated β-Catenin. (B and C) Relative β-Catenin/TCF-directed luciferase activity in ESCs (B) or neural stem cells (NSCs) (C) transfected with control siRNA or siRNA against Notch1 or Notch1-4. β-Catenin/TCF activity was measured by co-transfecting cells with a luciferase reporter downstream of multiple TCF binding sites (Topflash). (D) Relative RBP-J expression levels by qPCR in ESCs after transfection with control or RBP-J siRNA, determined by qPCR. (E) Western analysis of ESCs transfected with control or RBP-J siRNA (50 or 100 nM) with Act β-Cat antibodies. (F) Transverse sections (H&E) of control, Notch1 KO (Isl^{Cre}, Notch1^{tm2Rko}(ex3)^{loxP}) or RBP-J KO (Isl^{Cre}, RBP-J^{flox/flox}) embryos at embryonic day (ED) 9.5, at level of outflow tract (ot). Asterisks indicate precardiac mesoderm containing cardiac progenitor cells. The cutting plane is indicated by a dotted line (red) in an ED 9.5 embryo (left). All luciferase values were normalized to Renilla activity and represent n=4. *, P<0.01. Gapdh antibody was used as a loading control. h, head; ht, heart tube; Con, control; N1 KD, Notch1 siRNA; N1-4 KD, Notch 1-4 siRNA.

FIGS. 2A-J. Notch Negatively Regulates Active β-Catenin in ESCs by Physically Interacting with the RAM Domain (A) Relative expression of β-Catenin and Cyclin D1 mRNA in ESCs transfected with control or Notch1 siRNA (100 nM), determined by qPCR. (B) Western analysis of ESCs transfected with control or N1ICD (100 or 300 ng) and cultured with BIO. Gapdh antibody was used as a loading control. (C) Relative β-Catenin/TCF activity of BIO-treated ESCs transfected with control or N1ICD. (D) Relative β-Catenin/TCF activity of ESCs transfected with control or Notch1 siRNA and cultured with or without BIO. (E) Transverse sections (H&E) of control, Isl^{Cre}, β-catenin(ex3)^{loxP} (Act-β-Cat) or Isl^{Cre}, Gt(ROSA)26Sor^{tm1(Notch1)Dam}/J (Act-β-Cat; Notch1) embryos at ED 9.5, at level of outflow tract (ot). Boxed areas with asterisks indicate precardiac mesoderm containing cardiac progenitor cells. (F and G) ESCs treated with or without BIO (F) or SW480 cells (F and G) were transfected with expression constructs for Myc (−) or Myc-Notch1 intracellular domain (+), immunoprecipitated (IP) with anti-Myc antibody and immunoblotted (IB) with β-Catenin antibody recognizing its C-terminus (F), or dephosphorylated (active) form, or the phosphorylated N-terminus (G). Notch expression was detected with anti-Myc antibody (F). (H) Schematic representation of Notch1 deletion constructs and their interaction with β-Catenin. (I) Co-IP of BIO-treated ESCs with Notch1 deletion constructs shown in (H) using antibodies indicated. Arrowheads indicate Notch1 expression. (J) Relative β-Catenin/TCF activity of BIO-treated ESCs transfected with control or constructs shown in (H). TM (transmembrane domain), R (RAM domain), ANK (Ankyrin repeats), TA (transactivation domain), P (PEST domain). BIO was used at 2 μM. All luciferase values were normalized to Renilla activity and represent n=4. *, P<0.01. nt, neural tube; pe, pharyngeal endoderm. Con, control; N1, N1ICD; N1KD, Notch1 siRNA.

FIGS. 3A-I. Membrane-Bound Notch Negatively Regulates Active β-Catenin Levels in Stem Cells (A) Schematic representation of wildtype Notch1 and cleavage site-mutated tethered forms of Notch1 (V1744K and V1774L). (B) Relative β-Catenin/TCF activity of ESCs transfected with control, wildtype Notch1 (WT) or Notch1 mutants (V1744K and V1774L) shown in (A) and cultured with BIO. (C) BIO-treated ESCs transfected with WT or mutant Notch1 constructs and, immunoprecipitated (IP) with anti-Myc antibody and immunoblotted (IB) with β-Catenin antibody. Notch1 expression was detected with anti-Myc antibody. Arrowheads indicate cleaved Notch1. (D) Western analysis of active β-Catenin in ESCs transfected with WT or mutant Notch1 constructs. (E) Relative β-Catenin/TCF activity of ESCs and NSCs treated with increasing doses of DAPT for 72-96 h. (F) Western analysis of active β-Catenin in ESCs, NSCs, and bone marrow mesenchymal stem cells (MSCs) treated with increasing doses (0, 25, 50 or 100 μM) of DAPT for 72-96 h. (G) Relative number of ESCs after treatment with DAPT (50 or 100 μM) for 48 h. (H) Relative expression of Cyclin D1 mRNA in ESCs transfected with control (LacZ) or tethered Notch (V1774L) and cultured for 72 h. (I) Relative expression of Brachyury, Nestin and Sox17 mRNA in ESCs maintained without LIF for 72 h after transfection with control (LacZ) or tethered Notch (V1774L); *, P<0.01; NS, not significant. BIO was used at 2 μM. Con, control.

FIGS. 4A and 4B. Numb and Numb-like are required for Notch-mediated regulation of β-Catenin protein and activity (A) Relative β-Catenin/TCF activity of ESCs transfected with control (LacZ) or tethered Notch (V1774L) in the presence or absence of Numb/Numbl siRNA and cultured in BIO for 72 h (B) Western analysis of active β-Catenin in ESCs transfected with control (LacZ) or tethered Notch (V1774L) in the presence or absence of Numb/Numbl siRNA. All luciferase values were normalized to Renilla activity and represent n=4. Gapdh antibody was used as a loading control. *, P<0.01; NS, not significant. BIO was used at 2 μM. Con, control.

FIGS. 5A-I. γ-Secretase Inhibitors (GSIs) Suppress Expansion of Human Colon Cancer Cells by Blocking Notch Cleavage (A) Western analysis of active β-Catenin in SW480 colon cancer cells transfected with control or siRNA against Notch1-4 (100 nM each). (B) Relative β-Catenin/TCF activity of SW480 cells treated with increasing doses of DAPT for 96 h. (C) Western analysis of β-Catenin levels in SW480 and a second colon cancer cell line, HT-29, treated with increasing doses (0, 25, 50 or 100 μM) of DAPT for 96 h. (D) Relative number of SW480 cells treated with DAPT (50 or 100 μM) for 72 h. (E) Western analysis of active β-Catenin levels in SW480 cells with increasing DAPT in the presence or absence of proteasome inhibitor (PI) MG-132 (5 nM) for 72 h. Fewer PI-treated cells were loaded in the right panel since they exhibit higher levels of β-Catenin. (F) Notch/RBP-J activity of SW480 cells treated with increasing doses of Ibuprofen. Notch/RBP-J activity was measured by transfecting cells with a luciferase reporter downstream of multiple RBP-J sites. (G) Relative β-Catenin/TCF activity of SW480 cells treated with Ibuprofen for 72 h. (H) Western analysis of active β-Catenin in SW480 cells treated with Ibuprofen for 72 h. (I) Western analysis of active β-Catenin in SW480 cells transfected with Notch1-4 (100 nM each) siRNA and treated with or without ibuprofen. Gapdh antibody was used as a loading control. All luciferase values were normalized to Renilla activity and represent n=4. *, P<0.01. Con, control; N1-4 KD, Notch 1-4 siRNA.

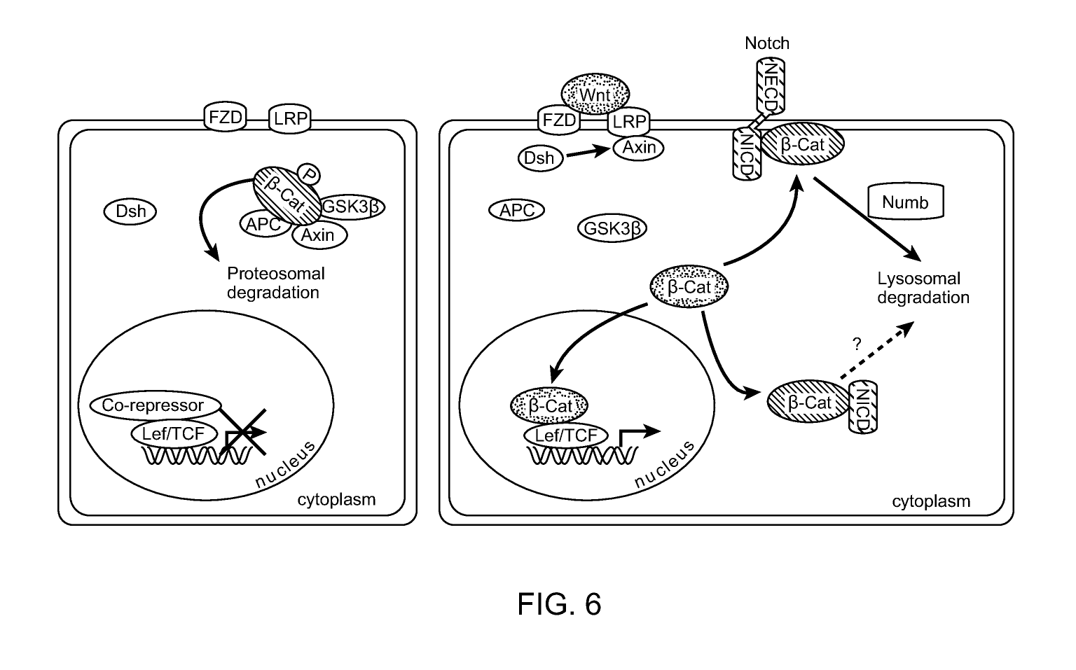

FIG. 6. Model for Post-Translational Regulation of β-Catenin Protein In the absence of Wnt, the destruction complex of Axin, APC and GSK313 phosphorylates β-Catenin, leading to its proteasomal degradation (left). When the destruction complex is inactivated by Wnts, unphosphorylated (active)β-Catenin functions as a transcriptional activator with TCF/LEF. We show that active β-Catenin protein levels can be negatively regulated by interaction with Notch in a Numb-dependent manner, possibly involving the lysosome. Notch-mediated degradation of β-Catenin is independent of the APC-dependent destruction complex. The cleaved Notch intracellular domain (NICD) can also interact with β-Catenin and lower its levels, but the mechanism of this and whether the NICD normally functions in this manner remains unknown.

### REFERENCES

Artavanis-Tsakonas, S., Rand, M. D., and Lake, R. J. (1999). Notch signaling: cell fate control and signal integration in development. Science 284, 770-776.
Bottone, F. G., Jr., Martinez, J. M., Collins, J. B., Afshari, C. A., and Eling, T. E. (2003). Gene modulation by the cyclooxygenase inhibitor, sulindac sulfide, in human colorectal carcinoma cells: possible link to apoptosis. The Journal of biological chemistry 278, 25790-25801.
Bu, L., Jiang, X., Martin-Puig, S., Caron, L., Zhu, S., Shao, Y., Roberts, D. J., Huang, P. L., Domian, I. J., and Chien, K. R. (2009). Human ISL1 heart progenitors generate diverse multipotent cardiovascular cell lineages. Nature 460, 113-117.
Cai, C. L., Liang, X., Shi, Y., Chu, P. H., Pfaff, S. L., Chen, J., and Evans, S. (2003). Isl1 identifies a cardiac progenitor population that proliferates prior to differentiation and contributes a majority of cells to the heart. Dev Cell 5, 877-889.
Chan, A. T., Giovannucci, E. L., Meyerhardt, J. A., Schernhammer, E. S., Curhan, G. C., and Fuchs, C. S. (2005). Long-term use of aspirin and nonsteroidal anti-inflammatory drugs and risk of colorectal cancer. Jama 294, 914-923.
Clevers, H. (2006). Wnt/beta-catenin signaling in development and disease. Cell 127, 469-480.
De Strooper, B., Annaert, W., Cupers, P., Saftig, P., Craessaerts, K., Mumm, J. S., Schroeter, E. H., Schrijvers, V., Wolfe, M. S., Ray, W. J., et al. (1999). A presenilin-1-dependent gamma-secretase-like protease mediates release of Notch intracellular domain. Nature 398, 518-522.
Eriksen, J. L., Sagi, S. A., Smith, T. E., Weggen, S., Das, P., McLendon, D. C., Ozols, V. V., Jessing, K. W., Zavitz, K. H., Koo, E. H., et al. (2003). NSAIDs and enantiomers of flurbiprofen target gamma-secretase and lower Abeta 42 in vivo. The Journal of clinical investigation 112, 440-449.
Fortini, M. E. (2009). Notch signaling: the core pathway and its posttranslational regulation. Dev Cell 16, 633-647.
Galceran, J., Sustmann, C., Hsu, S. C., Folberth, S., and Grosschedl, R. (2004). LEF1-mediated regulation of Delta-like1 links Wnt and Notch signaling in somitogenesis. Genes & development 18, 2718-2723.
Gulino, A., Di Marcotullio, L., and Screpanti, I. (2009). The multiple functions of Numb. Exp Cell Res.
Guo, M., Jan, L. Y., and Jan, Y. N. (1996). Control of daughter cell fates during asymmetric division: interaction of Numb and Notch. Neuron 17, 27-41.
Harada, N., Tamai, Y., Ishikawa, T., Sauer, B., Takaku, K., Oshima, M., and Taketo, M. M. (1999). Intestinal polyposis in mice with a dominant stable mutation of the beta-catenin gene. Embo J 18, 5931-5942.
Hayward, P., Kalmar, T., and Arias, A. M. (2008). Wnt/Notch signalling and information processing during development. Development 135, 411-424.
Kanwar, R., and Fortini, M. E. (2004). Notch signaling: a different sort makes the cut. Curr Biol 14, R1043-1045.
Koch, U., and Radtke, F. (2007). Notch and cancer: a double-edged sword. Cell Mol Life Sci 64, 2746-2762.
Kopan, R., and Ilagan, M. X. (2009). The canonical Notch signaling pathway: unfolding the activation mechanism. Cell 137, 216-233.
Korinek, V., Barker, N., Morin, P. J., van Wichen, D., de Weger, R., Kinzler, K. W., Vogelstein, B., and Clevers, H. (1997). Constitutive transcriptional activation by a beta-catenin-Tcf complex in APC−/− colon carcinoma. Science 275, 1784-1787.
Kwon, C., Arnold, J., Hsiao, E. C., Taketo, M. M., Conklin, B. R., and Srivastava, D. (2007). Canonical Wnt signaling is a positive regulator of mammalian cardiac progenitors. Proc Natl Acad Sci USA 104, 10894-10899.
Kwon, C., Qian, L., Cheng, P., Nigam, V., Arnold, J., and Srivastava, D. (2009). A regulatory pathway involving Notch1/beta-catenin/Isl1 determines cardiac progenitor cell fate. Nat Cell Biol 11, 951-957.
Logan, C. Y., and Nusse, R. (2004). The Wnt signaling pathway in development and disease. Annu Rev Cell Dev Biol 20, 781-810.
Lowell, S., Jones, P., Le Roux, I., Dunne, J., and Watt, F. M. (2000). Stimulation of human epidermal differentiation by delta-notch signalling at the boundaries of stem-cell clusters. Curr Biol 10, 491-500.
Lowry, W. E., Blanpain, C., Nowak, J. A., Guasch, G., Lewis, L., and Fuchs, E. (2005). Defining the impact of beta-catenin/Tcf transactivation on epithelial stem cells. Genes & development 19, 1596-1611.
Lu, H., and Bilder, D. (2005). Endocytic control of epithelial polarity and proliferation in Drosophila. Nature cell biology 7, 1232-1239.
Martinez Arias, A., Zecchini, V., and Brennan, K. (2002). CSL-independent Notch signalling: a checkpoint in cell fate decisions during development? Curr Opin Genet Dev 12, 524-533. McGill, M. A., Dho, S. E., Weinmaster, G., and McGlade, C. J. (2009). Numb regulates post-endocytic trafficking and degradation of Notch1. The Journal of biological chemistry 284, 26427-26438.
Meijer, L., Skaltsounis, A. L., Magiatis, P., Polychronopoulos, P., Knockaert, M., Leost, M., Ryan, X. P., Vonica, C. A., Brivanlou, A., Dajani, R., et al. (2003). GSK-3-selective inhibitors derived from Tyrian purple indirubins. Chem Biol 10, 1255-1266.
Murtaugh, L. C., Stanger, B. Z., Kwan, K. M., and Melton, D. A. (2003). Notch signaling controls multiple steps of pancreatic differentiation. Proc Natl Acad Sci USA 100, 14920-14925.
Peifer, M., and Polakis, P. (2000). Wnt signaling in oncogenesis and embryogenesis—a look outside the nucleus. Science (New York, N.Y. 287, 1606-1609.
Qyang, Y., Martin-Puig, S., Chiravuri, M., Chen, S., Xu, H., Bu, L., Jiang, X., Lin, L., Granger, A., Moretti, A., et al. (2007). The renewal and differentiation of Isl1+ cardiovascular progenitors are controlled by a Wnt/beta-catenin pathway. Cell stem cell 1, 165-179.
Rostom, A., Dube, C., Lewin, G., Tsertsvadze, A., Barrowman, N., Code, C., Sampson, M., and Moher, D. (2007). Nonsteroidal anti-inflammatory drugs and cyclooxygenase-2 inhibitors for primary prevention of colorectal cancer: a systematic review prepared for the U.S. Preventive Services Task Force. Ann Intern Med 146, 376-389.
Rusconi, J. C., and Corbin, V. (1998). Evidence for a novel Notch pathway required for muscle precursor selection in Drosophila. Mechanisms of development 79, 39-50.
Sanders, P. G., Munoz-Descalzo, S., Balayo, T., Wirtz-Peitz, F., Hayward, P., and Arias, A. M. (2009). Ligand-independent traffic of Notch buffers activated Armadillo in Drosophila. PLoS Biol 7, e1000169.
Sastre, M., Steiner, H., Fuchs, K., Capell, A., Multhaup, G., Condron, M. M., Teplow, D. B., and Haass, C. (2001). Presenilin-dependent gamma-secretase processing of beta-amyloid precursor protein at a site corresponding to the S3 cleavage of Notch. EMBO Rep 2, 835-841.
Sato, N., Meijer, L., Skaltsounis, L., Greengard, P., and Brivanlou, A. H. (2004). Maintenance of pluripotency in human and mouse embryonic stem cells through activation of Wnt signaling by a pharmacological GSK-3-specific inhibitor. Nature medicine 10, 55-63.
Schroeter, E. H., Kisslinger, J. A., and Kopan, R. (1998). Notch-1 signalling requires ligand-induced proteolytic release of intracellular domain. Nature 393, 382-386.
Shiff, S. J., Qiao, L., Tsai, L. L., and Rigas, B. (1995). Sulindac sulfide, an aspirin-like compound, inhibits proliferation, causes cell cycle quiescence, and induces apoptosis in HT-29 colon adenocarcinoma cells. The Journal of clinical investigation 96, 491-503.
Srinivas, S., Watanabe, T., Lin, C. S., William, C. M., Tanabe, Y., Jessell, T. M., and Costantini, F. (2001). Cre reporter strains produced by targeted insertion of EYFP and ECFP into the ROSA26 locus. BMC Dev Biol 1, 4.
Tamura, K., Taniguchi, Y., Minoguchi, S., Sakai, T., Tun, T., Furukawa, T., and Honjo, T. (1995). Physical interaction between a novel domain of the receptor Notch and the transcription factor RBP-J kappa/Su(H). Curr Biol 5, 1416-1423.
Tanigaki, K., Han, H., Yamamoto, N., Tashiro, K., Ikegawa, M., Kuroda, K., Suzuki, A., Nakano, T., and Honjo, T. (2002). Notch-RBP-J signaling is involved in cell fate determination of marginal zone B cells. Nat Immunol 3, 443-450.
Tetsu, O., and McCormick, F. (1999). Beta-catenin regulates expression of cyclin D1 in colon carcinoma cells. Nature 398, 422-426.
Tien, A. C., Rajan, A., and Bellen, H. J. (2009). A Notch updated. J Cell Biol 184, 621-629.
van Es, J. H., van Gijn, M. E., Riccio, O., van den Born, M., Vooijs, M., Begthel, H., Cozijnsen, M., Robine, S., Winton, D. J., Radtke, F., et al. (2005). Notch/gamma-secretase inhibition turns proliferative cells in intestinal crypts and adenomas into goblet cells. Nature 435, 959-963.
Winston, J. T., Strack, P., Beer-Romero, P., Chu, C. Y., Elledge, S. J., and Harper, J. W. (1999). The SCFbeta-TRCP-ubiquitin ligase complex associates specifically with phosphorylated destruction motifs in IkappaBalpha and beta-catenin and stimulates IkappaBalpha ubiquitination in vitro. Genes Dev 13, 270-283.
Yamamoto, N., Yamamoto, S., Inagaki, F., Kawaichi, M., Fukamizu, A., Kishi, N., Matsuno, K., Nakamura, K., Weinmaster, G., Okano, H., et al. (2001). Role of Deltex-1 as a transcriptional regulator downstream of the Notch receptor. J Biol Chem 276, 45031-45040.
Yang, X., Klein, R., Tian, X., Cheng, H. T., Kopan, R., and Shen, J. (2004). Notch activation induces apoptosis in neural progenitor cells through a p53-dependent pathway. Dev Biol 269, 81-94.
Yu, X., Zou, J., Ye, Z., Hammond, H., Chen, G., Tokunaga, A., Mali, P., Li, Y. M., Civin, C., Gaiano, N., et al. (2008). Notch signaling activation in human embryonic stem cells is required for embryonic, but not trophoblastic, lineage commitment. Cell stem cell 2, 461-471.
Zhong, W., Feder, J. N., Jiang, M. M., Jan, L. Y., and Jan, Y. N. (1996). Asymmetric localization of a mammalian numb homolog during mouse cortical neurogenesis. Neuron 17, 43-53.
Zhong, W., Jiang, M. M., Weinmaster, G., Jan, L. Y., and Jan, Y. N. (1997). Differential expression of mammalian Numb, Numblike and Notch1 suggests distinct roles during mouse cortical neurogenesis. Development 124, 1887-1897.
Zhu, A. J., and Watt, F. M. (1999). beta-catenin signalling modulates proliferative potential of human epidermal keratinocytes independently of intercellular adhesion. Development 126, 2285-2298.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for increasing self-renewal or expansion of a stem cell, the method comprising contacting the stem cell with an agent that inhibits binding between the intracellular domain of a membrane-bound Notch1 polypeptide and a β-catenin polypeptide.

2. The method of claim 1, wherein the agent is a polypeptide fragment of Notch1 that competes with full-length Notch1 for binding to β-catenin, wherein the Notch1 fragment does not induce degradation of β-catenin.

3. The method of claim 2, wherein the Notch1 fragment has a length of 100 amino acids or less and comprises an amino acid sequence having at least about 85% amino acid sequence identity to a contiguous stretch of at least 25 amino acids of amino acids 1759-2556 of the amino acid sequence set forth in SEQ ID NO:1.

4. The method of claim 3, wherein the Notch1 fragment has a length of from about 20 amino acids to about 100 amino acids.

5. The method of claim 2, wherein the Notch1 fragment is cyclized.

6. The method of claim 2, wherein the Notch1 fragment comprises a protein transduction domain.

7. The method of claim 1, wherein the agent is an expression vector comprising a nucleotide sequence encoding a polypeptide fragment of Notch1 that competes with full-length Notch1 for binding to β-catenin, wherein the Notch1 fragment does not induce degradation of β-catenin.

8. The method of claim 7, wherein the nucleotide sequence is operably linked to a constitutive promoter or an inducible promoter.

9. The method of claim 1, wherein said contacting is carried out in vitro.

10. A method of reducing uncontrolled cell proliferation, the method comprising contacting a cell that exhibits uncontrolled cell proliferation with an agent that inhibits cleavage of an intracellular portion of a membrane-bound Notch1 polypeptide from the transmembrane domain of the membrane-bound Notch1 polypeptide or that stabilizes binding of membrane-bound Notch1 polypeptide to β-catenin via the intracellular domain of the membrane-bound Notch1.

11. The method of claim 10, wherein the agent is a γ-secretase inhibitor.

12. The method of claim 11, wherein the γ-secretase inhibitor selectively inhibits cleavage of an intracellular portion of a membrane-bound Notch1 polypeptide from the transmembrane domain of the membrane-bound Notch1 polypeptide.

13. The method of claim 10, wherein the cell that exhibits uncontrolled cell proliferation is a cancer cell.

14. An in vitro method for identifying an agent that blocks binding of an intracellular domain of a Notch1 polypeptide to β-catenin, the method comprising:
a) contacting a Notch1 polypeptide that comprises the intracellular domain of a Notch1 polypeptide with a test agent and a β-catenin polypeptide; and
b) determining the effect, if any, of the test agent on binding of the Notch1 polypeptide to the β-catenin polypeptide, wherein a test agent that reduces binding of the Notch1 polypeptide to the β-catenin polypeptide by at least about 10% is a candidate agent for increasing stem cell self-renewal and/or expansion.

15. The method of claim 14, wherein said β-catenin polypeptide comprises an amino acid sequence having at least about 85% amino acid sequence identity to a contiguous stretch of at least 25 amino acids of the amino acid sequence set forth in SEQ ID NO:3.

16. The method of claim 14, wherein said contacting and determining are carried out in a cell-free assay.

17. The method of claim 14, wherein the Notch1 polypeptide comprises an amino acid sequence having at least about 85% amino acid sequence identity to a contiguous stretch of at least 25 amino acids of amino acids 1759-2556 of the amino acid sequence set forth in SEQ ID NO:1.

18. The method of claim 14, wherein said determining is carried out using a protein blot assay, an enzyme-linked immunosorbent assay, a BRET assay, a FRET assay, or an immunoprecipitation assay.

19. The method of claim 14, wherein one or both of the Notch1 polypeptide and the β-catenin polypeptide comprises a detectable label.

20. The method of claim 14, wherein one or both of the Notch1 polypeptide and the β-catenin polypeptide is a fusion protein comprising a fusion partner.

21. An in vitro method of identifying an agent that increases binding of β-catenin to an intracellular domain of a Notch1 polypeptide, the method comprising:
a) contacting a Notch1 polypeptide that comprises the intracellular domain of a Notch1 polypeptide with a test agent and a β-catenin polypeptide; and
b) determining the effect, if any, of the test agent on binding of the Notch1 polypeptide to the β-catenin polypeptide, wherein a test agent that increases binding of β-catenin to an intracellular domain of a Notch1 polypeptide is a candidate agent for reducing cell proliferation.

22. An in vitro method of identifying an agent that reduces cleavage of the intracellular domain of a Notch1 polypeptide from the transmembrane domain of the Notch1 polypeptide, the method comprising:
a) contacting a Notch1 polypeptide that comprises the transmembrane domain and the intracellular domain of a Notch1 polypeptide with a test agent and an enzyme that cleaves the intracellular domain of the Notch1 polypeptide from the transmembrane domain of the Notch1 polypeptide; and
b) determining the effect, if any, of the test agent on cleavage of the intracellular domain of the Notch1 polypeptide from the transmembrane domain of the Notch1 polypeptide mediated by the enzyme, wherein an agent that reduces the cleavage by at least about 10% is considered a candidate agent for reducing cell proliferation.

23. The method of claim 22, wherein the enzyme is a γ-secretase.

24. The method of claim 22, wherein the assay is carried out in a cell-free assay system.

25. The method of claim 22, wherein the Notch1 polypeptide comprises an amino acid sequence having at least about 85% amino acid sequence identity to amino acids 1737-2556 of the amino acid sequence set forth in SEQ ID NO:1.

26. The methods of claim 22, wherein the Notch1 polypeptide lacks extracellular domains.
